(19) United States
(12) Patent Application Publication
(10) Pub. No.: US 2024/0398038 A1
(43) Pub. Date: Dec. 5, 2024

(54) AEROSOL-GENERATING DEVICES AND METHODS FOR GENERATING AEROSOL

(71) Applicant: Altria Client Services LLC, Richmond, VA (US)

(72) Inventors: Nam TRAN, North Chesterfield, VA (US); Eric HAWES, Glen Allen, VA (US); Zack W. BLACKMON, Richmond, VA (US); Terrance T. BACHE, Richmond, VA (US); Rangaraj S. SUNDAR, Midlothian, VA (US); Raymond W. LAU, Glen Allen, VA (US); Niall GALLAGHER, Cambridge (GB)

(73) Assignee: Altria Client Services LLC, Richmond, VA (US)

(21) Appl. No.: 18/732,733

(22) Filed: Jun. 4, 2024

Related U.S. Application Data
(60) Provisional application No. 63/506,242, filed on Jun. 5, 2023.

(51) Int. Cl. A24F 40/57; A24F 40/42; A24F 40/51; A61M 15/06; H05B 1/02

(52) U.S. Cl. CPC A24F 40/57; A24F 40/42; A24F 40/51; H05B 1/0244; A61M 15/06

(57) ABSTRACT

An aerosol-generating device includes a sensor configured to detect puffs; and processing circuitry configured to cause the aerosol-generating device to increase a temperature of a heater to a preheat temperature, decrease the temperature of the heater to a first heating temperature before a first detected puff of the detected puffs, the first heating temperature being below the preheat temperature, and increase the temperature of the heater based on a number of the detected puffs.

### CROSS-REFERENCE TO RELATED APPLICATION

The present application claims priority to U.S. Provisional Application No. 63/506,242, filed Jun. 5, 2023, the entire contents of which are hereby incorporated by reference.

### BACKGROUND

### Field

The present disclosure relates to aerosol-generating devices and methods for generating aerosol.

### Description of Related Art

Some electronic devices are configured to heat a plant material to a temperature that is sufficient to release constituents of the plant material while keeping the temperature below a combustion point of the plant material so as to avoid any substantial pyrolysis of the plant material. Such devices may be referred to as aerosol-generating devices (e.g., heated tobacco aerosol-generating devices), and the plant material heated may be tobacco and/or cannabis. In some instances, the plant material may be introduced directly into a heating chamber of an aerosol-generating device. In other instances, the plant material may be pre-packaged in individual containers to facilitate insertion and removal from an aerosol-generating device.

### SUMMARY

At least some example embodiments relate to an aerosol-generating device.

According to at least one example embodiment, an aerosol-generating device includes a sensor configured to detect puffs; and processing circuitry configured to cause the aerosol-generating device to increase a temperature of a heater to a preheat temperature, decrease the temperature of the heater to a first heating temperature before a first detected puff of the detected puffs, the first heating temperature being below the preheat temperature, and increase the temperature of the heater based on a number of the detected puffs.

According to at least one example embodiment, each puff of the at least one detected puff is associated with a temperature setpoint.

According to at least one example embodiment, each temperature setpoint associated with a puff is greater than or equal to a temperature setpoint associated with a previous puff.

According to at least one example embodiment, at least two sequential puffs are associated with the same temperature setpoint.

According to at least one example embodiment, the processing circuitry is configured to cause the aerosol-generating device to increase the temperature of the heater to a second heating temperature after a first number of the at least one detected puff, the first number being greater than one.

According to at least one example embodiment, the second heating temperature is greater than the first heating temperature and less than the preheat temperature.

According to at least one example embodiment, the at least one detected puff is a plurality of detected puffs and the processing circuitry is configured to cause the aerosol-generating device to maintain the first heating temperature until a second number of the plurality of detected puffs and increase the temperature of the heater to a third heating temperature after the second number of the plurality of detected puffs, the second number of the plurality of detected puffs being greater than two.

According to at least one example embodiment, the processing circuitry is configured to cause the aerosol-generating device to increase the temperature of the heater when the number of the at least one detected puff reaches a predetermined number.

According to at least one example embodiment, the processing circuitry is configured to cause the aerosol-generating device to increase the temperature of the heater to a final heating temperature when the number of the at least one detected puff reaches the predetermined number.

According to at least one example embodiment, the processing circuitry is configured to cause the aerosol-generating device to maintain the temperature of the heater at the final heating temperature until the number of the at least one detected puff reaches a maximum number.

According to at least one example embodiment, the final heating temperature is less than or equal to the preheat temperature.

According to at least one example embodiment, the final heating temperature is greater than the preheat temperature.

According to at least one example embodiment, the processing circuitry is configured to cause the aerosol-generating device to reduce a power to the heater to a first power during at least one of the detected puffs.

According to at least one example embodiment, the at least one detected puff is a plurality of detected puffs and the processing circuitry is configured to cause the aerosol-generating device to supply a second power to the heater for a period of time between adjacent puffs of the plurality of detected puffs, the second power being greater than the first power.

According to at least one example embodiment, the processing circuitry is configured to cause the aerosol-generating device to supply the second power to the heater for the period of time using a proportional-integral-derivative (PID) controller, wherein the processing circuitry is configured to cause the aerosol-generating device to change at least one of a proportional term, an integral term and a derivative term of the PID controller.

According to at least one example embodiment, the processing circuitry is configured to cause the aerosol-generating device to maintain values of the proportional term, the integral term and the derivative term of the PID controller during the period of time.

According to at least one example embodiment, the processing circuitry is configured to cause the aerosol-generating device to determine a voltage applied to the heater and a current applied to the heater over a period of time, and decrease the temperature of the heater to the first heating temperature based on the voltage applied to the heater and the current applied to the heater over the period of time.

According to at least one example embodiment, the processing circuitry is configured to cause the aerosol-generating device to determine a sum of products of the voltage applied to the heater and the current applied to the heater, and determine if the sum is greater than a threshold, wherein the decrease of the temperature of the heater to the first heating temperature occurs when the sum is greater than the threshold.

According to at least one example embodiment, the aerosol-generating device is configured to receive a capsule containing an aerosol-forming substrate to be heated by the heater.

According to at least one example embodiment, the heater is in the capsule.

According to at least one example embodiment, the heater is external to the capsule.

Accordingly to at least one example embodiment, a method of generating aerosol in an aerosol-generating device includes increasing a temperature of a heater of the aerosol-generating device to a preheat temperature; decreasing the temperature of the heater to a first heating temperature; detecting at least one puff after decreasing the temperature of the heater to the first heating temperature; and increasing the temperature of the heater based on a number of the at least one detected puff.

### BRIEF DESCRIPTION OF THE DRAWINGS

The various features and advantages of the non-limiting embodiments herein may become more apparent upon review of the detailed description in conjunction with the accompanying drawings. The accompanying drawings are merely provided for illustrative purposes and should not be interpreted to limit the scope of the claims. The accompanying drawings are not to be considered as drawn to scale unless explicitly noted. For purposes of clarity, various dimensions of the drawings may have been exaggerated.

FIG. 1 is a top right, front perspective view of an example aerosol-generating device in accordance with at least one example embodiment.

FIG. 2 is a bottom right, front perspective view of the example aerosol-generating device illustrated in FIG. 1.

FIG. 3 is a bottom view of the example aerosol-generating device illustrated in FIG. 1.

FIG. 4 is a top right, front perspective view of the example aerosol-generating device illustrated in FIG. 1, including a capsule where the lid is opened.

FIG. 5 is a cross-sectional view of the example aerosol-generating device illustrated in FIG. 4.

FIG. 6 is a partial cross-sectional view of the example capsule connector illustrated in FIG. 5.

FIG. 7 is a partial front perspective view of the example aerosol-generating device illustrated in FIG. 4, where a section of the housing has been removed.

FIG. 8 illustrates electrical systems of an aerosol-generating device and a capsule according to one or more example embodiments.

FIG. 9 illustrates a heater voltage measurement circuit according to one or more example embodiments.

FIG. 10 illustrates a heater current measurement circuit according to one or more example embodiments.

FIG. 11 illustrates a compensation voltage measurement circuit according to one or more example embodiments.

FIGS. 12A-12C illustrates circuit diagrams of a heating engine control circuit according to one or more example embodiments.

FIGS. 13A-13C illustrate methods of controlling a heater in a non-combustible aerosol-generating device according to one or more example embodiments.

FIG. 14 illustrates a block diagram illustrating a temperature heating engine control algorithm according to one or more example embodiments.

FIG. 15A illustrates a temperature profile of a heater according to a puff count according to one or more example embodiments.

FIG. 15B illustrates a power profile overlaid on the temperature profile shown in FIG. 15A.

FIG. 16A illustrates a temperature profile of a heater according to a puff count according to one or more example embodiments.

FIG. 16B illustrates a power profile overlaid on the temperature profile shown in FIG. 15A.

### DETAILED DESCRIPTION OF EXAMPLE EMBODIMENTS

Some detailed example embodiments are disclosed herein. However, specific structural and functional details disclosed herein are merely representative for purposes of describing example embodiments. Example embodiments may, however, be embodied in many alternate forms and should not be construed as limited to only the example embodiments set forth herein.

Accordingly, while example embodiments are capable of various modifications and alternative forms, example embodiments thereof are shown by way of example in the drawings and will herein be described in detail. It should be understood, however, that there is no intent to limit example embodiments to the particular forms disclosed, but to the contrary, example embodiments are to cover all modifications, equivalents, and alternatives falling within the scope of example embodiments. Like numbers refer to like elements throughout the description of the figures.

It should be understood that when an element or layer is referred to as being “on,” “connected to,” “coupled to,” or “covering” another element or layer, it may be directly on, connected to, coupled to, or covering the other element or layer or intervening elements or layers may be present. In contrast, when an element is referred to as being “directly on,” “directly connected to,” or “directly coupled to” another element or layer, there are no intervening elements or layers present. Like numbers refer to like elements throughout the specification. As used herein, the term “and/or” includes any and all combinations of one or more of the associated listed items.

It should be understood that, although the terms first, second, third, etc. may be used herein to describe various elements, regions, layers and/or sections, these elements, regions, layers, and/or sections should not be limited by these terms. These terms are only used to distinguish one element, region, layer, or section from another region, layer, or section. Thus, a first element, component, region, layer, or section discussed below could be termed a second element, region, layer, or section without departing from the teachings of example embodiments.

Spatially relative terms (e.g., “beneath,” “below,” “lower,” “above,” “upper,” and the like) may be used herein for ease of description to describe one element or feature's relationship to another element(s) or feature(s) as illustrated in the figures. It should be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” may encompass both an orientation of above and below. The device may be otherwise oriented (rotated 90 degrees or at other orientations) and the spatially relative descriptors used herein interpreted accordingly.

The terminology used herein is for the purpose of describing various example embodiments only and is not intended to be limiting of example embodiments. As used herein, the singular forms “a,” “an,” and “the” are intended to include the plural forms as well, unless the context clearly indicates otherwise. It will be further understood that the terms “includes,” “including,” “comprises,” and/or “comprising,” specify the presence of stated features, integers, steps, operations, and/or elements, but do not preclude the presence or addition of one or more other features, integers, steps, operations, elements, and/or groups thereof.

When the terms “about” or “substantially” are used in this specification in connection with a numerical value, it is intended that the associated numerical value includes a manufacturing or operational tolerance (e.g., ±10%) around the stated numerical value. Moreover, when the terms “generally” or “substantially” are used in connection with geometric shapes, it is intended that precision of the geometric shape is not required but that latitude for the shape is within the scope of the disclosure. Furthermore, regardless of whether numerical values or shapes are modified as “about,” “generally,” or “substantially,” it will be understood that these values and shapes should be construed as including a manufacturing or operational tolerance (e.g., ±10%) around the stated numerical values or shapes.

Unless otherwise defined, all terms (including technical and scientific terms) used herein have the same meaning as commonly understood by one of ordinary skill in the art to which example embodiments belong. It will be further understood that terms, including those defined in commonly used dictionaries, should be interpreted as having a meaning that is consistent with their meaning in the context of the relevant art and will not be interpreted in an idealized or overly formal sense unless expressly so defined herein.

As used herein, “coupled” includes both removably coupled and permanently coupled. For example, when an elastic layer and a support layer are removably coupled to one another, the elastic layer and the support layer can be separated upon the application of sufficient force.

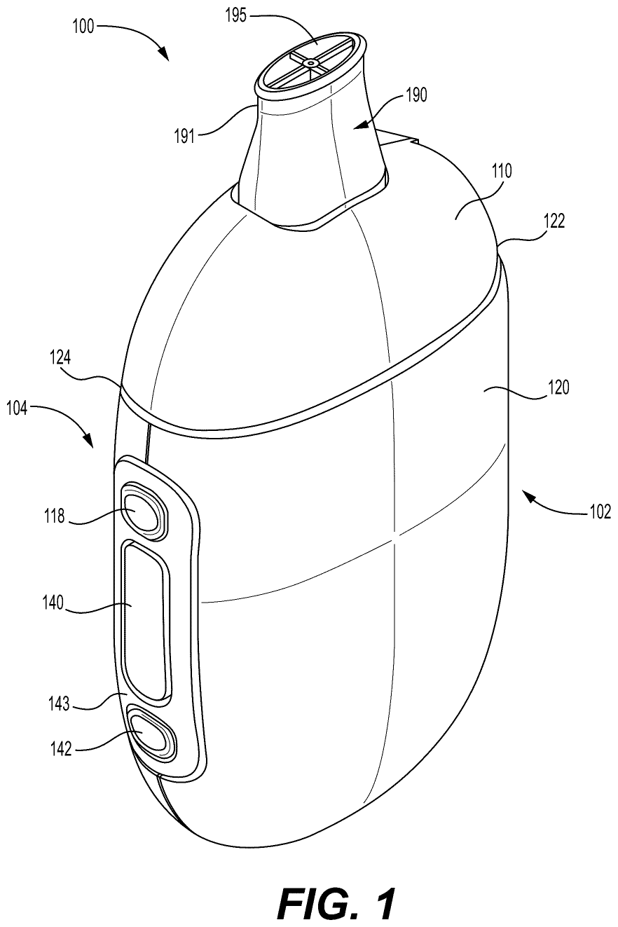

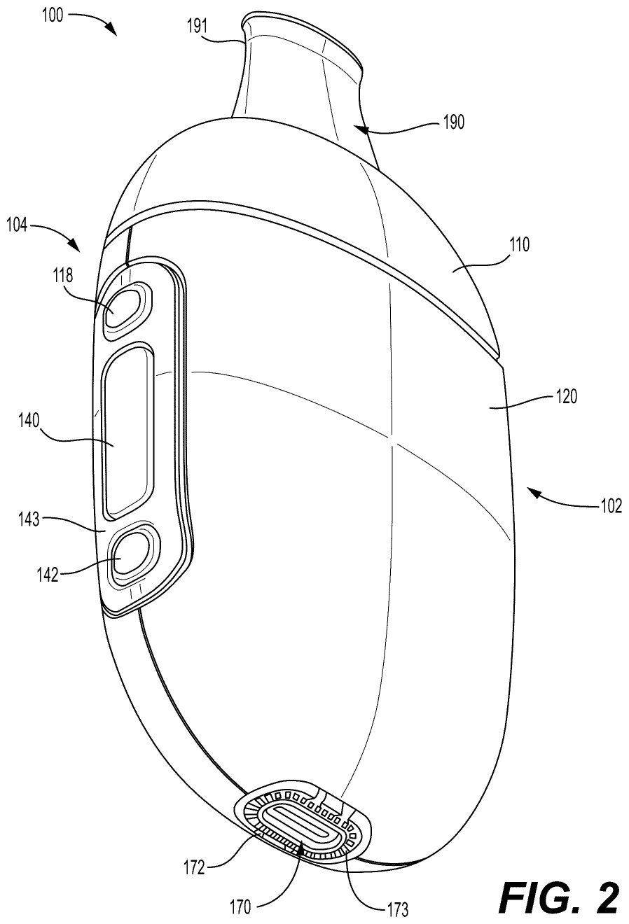

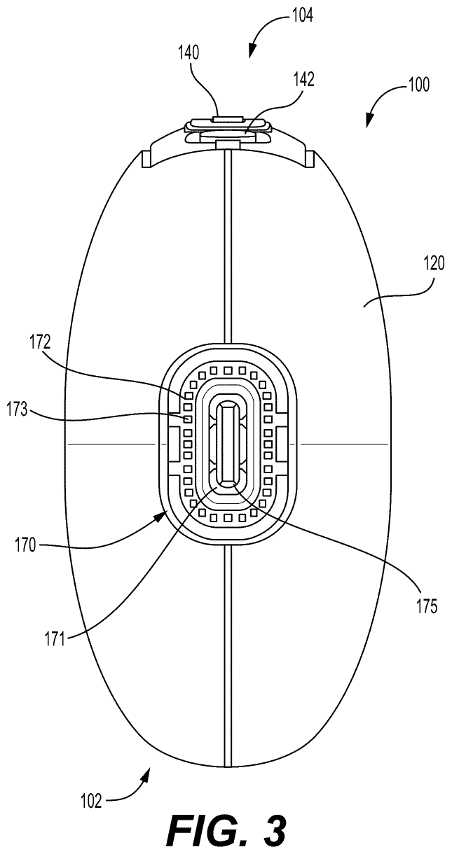

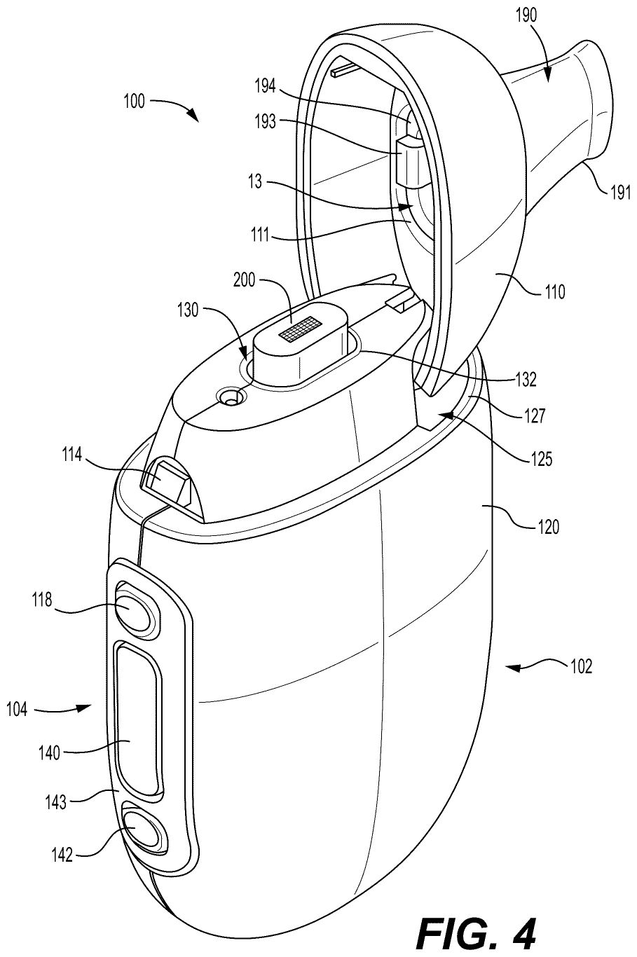

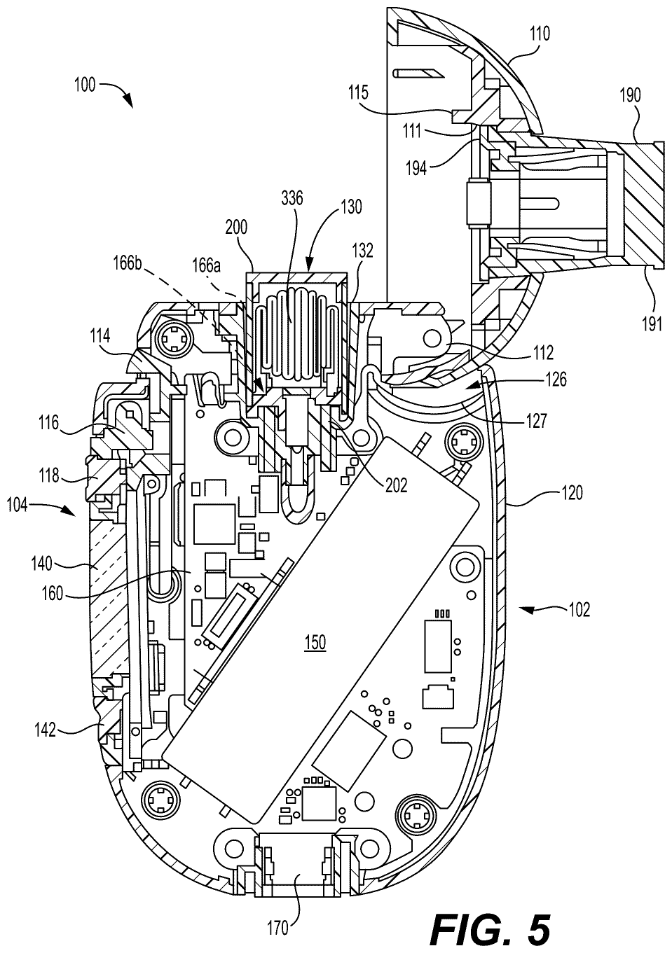

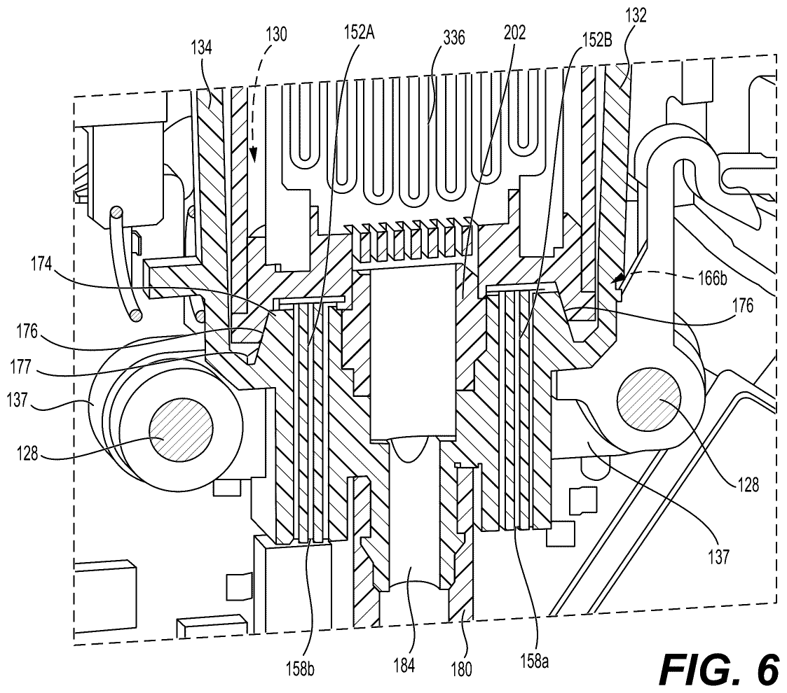

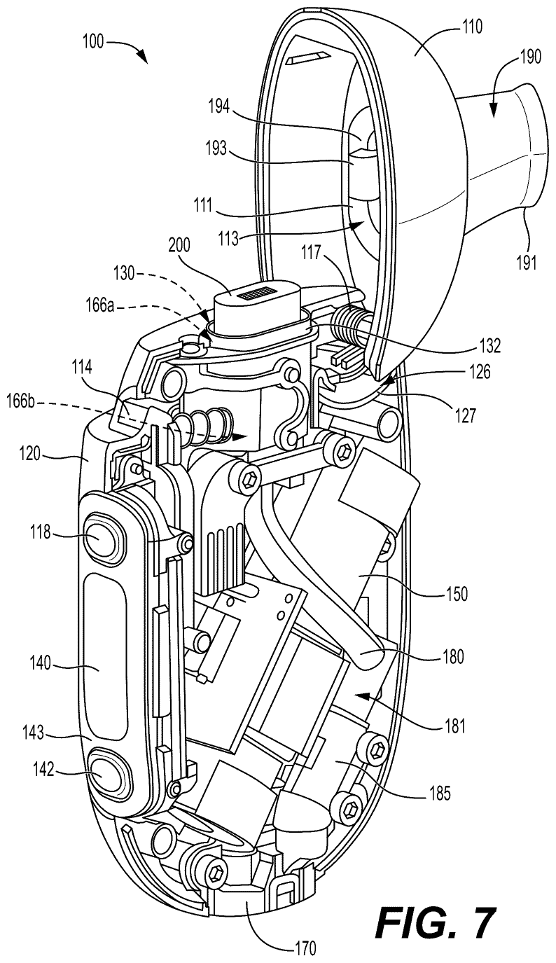

FIGS. 1-7 are illustrations of an aerosol-generating device 100 (e.g., a heated tobacco aerosol-generating device) in accordance with at least one example embodiment. For example, FIG. 1 is a top perspective view of the aerosol-generating device 100, where the lid 110 is in a closed position. FIG. 2 is a bottom perspective view of the aerosol-generating device 100, where the lid 110 is in a closed position. FIG. 3 is a bottom-up view of the aerosol-generating device 100, where the lid 110 is in a closed position. FIG. 4 is another top perspective view of the aerosol-generating device 100, where the lid 110 is opened and a capsule 200 is received by a capsule receiving cavity 130. FIG. 5 is a cross-sectional view of the aerosol-generating device 100, where the lid 110 is opened and a capsule 200 is received by the capsule receiving cavity 130. FIG. 6 is a partial cross-sectional view of a capsule connector. FIG. 7 is a partial, perspective view of the aerosol-generating device 100, where a section of the housing 120 has been removed to show various internal components, the lid 110 is opened, and a capsule 200 is received by the capsule receiving cavity 130.

As illustrated, in at least one example embodiment, the aerosol-generating device 100 has a general oval or oblong or pebble shape and a replaceable mouthpiece 190 that extends from the main body of the aerosol-generating device 100. For example, the aerosol-generating device 100 may include a housing 120 that defines a capsule-receiving cavity 130 (as best shown in FIG. 4-5) and a lid 110 that is configured to open/close relative to the housing 120 and is coupleable to the replaceable mouthpiece 190. For example, the lid 110 may be fixedly coupled to the housing 120 at a first point 122 and releasably coupleable to the housing 120 at a second point 124. The first point 122 of the housing 120 may be on a first side 102 of the aerosol-generating device 100, while the second point 124 of the housing 120 may be on a second side 104 of the aerosol-generating device 100. In some instances, the lid 110 may also be referred to as a door.

An exterior of the housing 120 and/or lid 110 may be formed from a metal (such as aluminum, stainless steel, and the like); an aesthetic, food contact rated plastic (such as, a polycarbonate (PC), acrylonitrile butadiene styrene (ABS) material, liquid crystalline polymer (LCP), a copolyester plastic, or any other suitable polymer and/or plastic); or any combination thereof. The replaceable mouthpiece 190 may be similarly formed from a metal (such as aluminum, stainless steel, and the like); an aesthetic, food contact rated plastic (such as, a polycarbonate (PC), acrylonitrile butadiene styrene (ABS) material, liquid crystalline polymer (LCP), a copolyester plastic, or any other suitable polymer and/or plastic); and/or plant-based materials (such as wood, bamboo, and the like). One or more interior surfaces of the housing 120 and/or lid 110 may be formed from or coated with a high temperature plastic (such as, polyetheretherketone (PEEK), liquid crystal polymer (LCP), or the like). The lid 110 and the housing 120 may be collectively regarded as the main body of the aerosol-generating device 100.

The lid 110 may be fixedly coupled to the housing 120 at the first point 122 by a hinge 112, or other similar connector, that allows the lid 110 to move (e.g., swing and rotate) from an open position (such as illustrated in FIGS. 4-5) to a closed position (such as illustrated in FIG. 1-2). As illustrated in FIG. 7, the hinge 112 may include a torsion spring 117. In at least one example embodiment, such as illustrated in FIG. 5, the housing 120 includes a recess 126 at the first point 122. The recess 126 may be configured to receive a portion of the lid 110 so as to allow for an easy and smooth movement of the lid 110 from the open position to the closed position (and vice versa). The recess 126 may have a structure that corresponds with a relative portion of the lid 110. For example, as illustrated, the recess 126 may include a substantially curved portion 127 that has a general concave shape that corresponds with the curvature of the lid 110, which has a general convex shape.

The lid 110 may be releasably coupleable to the housing 120 at the second point 124 by a latch 114, or other similar connector, that allows the lid 110 to be fixed or secured in the closed position and easily releasable so as to allow the lid 110 to move from the secured closed position to the open position. In at least one example embodiment, the latch 114 may be coupled to a latch release mechanism 116. The latch release mechanism 116 may be configured to move the latch 114 from a first or closed position to a second or open position. For example, such as illustrated in FIG. 5, the latch 114 may extend downwards in the housing 120 and the latch release mechanism 116 may be perpendicular to the downwards length of the latch 114. As such, the latch release mechanism 116 is configured to apply pressure to the latch 114. For example, the latch release mechanism 116 may be movable between a first position and a second position. In the first position, the latch release mechanism 116 may be neutral relative to the latch 114. In the second position, the latch release mechanism 116 may apply pressure to the downwards length of the latch 114 so as to move the latch 114 from the secured or latched close position to the open position.

In at least one example embodiment, such as best illustrated in FIG. 5, the latch release mechanism 116 is in communication with a latch release button 118 that is configured to activate the latch release mechanism 116—i.e., to move the latch 114 from the first or closed or secured position to the second or pressure-applying position and to move/return the latch 114 from the open position to the secured or closed position. In at least one example embodiment, the latch release button 118 is an adult consumer interaction button disposed on the second side 104 of the aerosol-generating device 100. For example, when the latch release button 118 is pressed by the adult consumer, the latch release mechanism 116 may move from the first or closed or secured position to the second or pressure-applying position so as to move the latch 114 from the secured or closed position to the open position. The latch release button 118 may have a substantially circular shape with a center depression or dimple configured to direct the pressure applied by the adult consumer, although example embodiments are not limited thereto. One or more sensors (not shown) configured to detect the lid 110 opening and closure may be embedded or otherwise disposed within the housing 120 and/or one or more of the elements therein (e.g., latch 114, latch release mechanism 116, latch release button 118).

In at least one example embodiment, such as illustrated in FIGS. 1-2, the housing 120 includes a consumer interface panel 143 disposed on the second side 104 of the aerosol-generating device 100. For example, the consumer interface panel 143 may be an oval-shaped panel that runs along the second side 104 of the aerosol-generating device 100. The consumer interface panel 143 may include the latch release button 118, such as discussed above, as well as a communication screen 140 and/or a power button 142. For example, in at least one example embodiment, the consumer interface panel 143 may include the communication screen 140 disposed between the latch release button 118 and the power button 142. As illustrated, the latch release button 118 may be disposed towards a top of the aerosol-generating device 100, and the power button 142 may be disposed towards bottom of the aerosol-generating device 100. Like the latch release button 118, the power button 142 may also be an adult consumer interaction button. The power button 142 may have a substantially circular shape with a center depression or dimple configured to direct the pressure applied by the adult consumer, although example embodiments are not limited thereto. The power button 142 may turn on and off the aerosol-generating device 100. Though only the two buttons are illustrated, it should be understood more or less buttons may be provided depending on the available features and desired adult consumer interface.

In at least one example embodiment, the communication screen 140 is an integrated thin-film transistor (“TFT”) screen. In other example embodiments, the communication screen 140 is an organic light emitting diode (“OLED”) or light emitting diode (“LED”) screen. The communication screen 140 is configured for adult consumer engagement and may have a generally oblong shape.

In at least one example embodiment, the housing 120 defines a charging connector or port 170. For example, as best illustrated in FIG. 2, the charging connector 170 may be defined/disposed in a bottom or second end of the housing 120 distal from the capsule-receiving cavity 130. The charging connector 170 may be configured to receive an electric current (e.g., via a USB/mini-USB cable) from an external power source so as to charge the power source 150 internal to the aerosol-generating device 100. For example, in at least one example embodiment, such as best illustrated in FIG. 3, the charging connector 170 may be an assembly defining a cavity 171 that has a projection 175 within the cavity 171. In at least one example embodiment, the projection 175 does not extend beyond the rim of the cavity 171. In addition, the charging connector 170 may also be configured to send data to and/or receive data (e.g., via a USB/mini-USB cable) from another aerosol-generating device (e.g., a heated tobacco aerosol-generating device) and/or other electronic device (e.g., phone, tablet, computer, and the like). In at least one embodiment, the aerosol-generating device 100 may instead or additionally be configured for wireless communication (e.g., via Bluetooth) with such other aerosol-generating devices and/or electronic devices.

In at least one example embodiment, such as best illustrated in FIG. 3, a protective grille 172 is disposed around the charging connector 170. The protective grille 172 may be configured to help reduce or prevent debris ingress and/or the inadvertent blockage of the incoming airflow. For example, the protective grille 172 may define a plurality of pores 173 along its length or course. As illustrated, the protective grille 172 may have an annular form that surrounds the charging connector 170. In this regard, the pores 173 may also be arranged (e.g., in a serial arrangement) around the charging connector 170. Each of the pores 173 may have an oval or circular shape, although not limited thereto. In at least one example embodiment, the protective grille 172 may include an approved food contact material. For example, the protective grille 172 may include plastic, metal (e.g., stainless steel, aluminum), or any combination thereof. In at least one example embodiment, a surface of the protective grille 172 may be coated, for example with a thin layer of plastic, and/or anodized.

The pores 173 in the protective grille 172 may function as inlets for air drawn into the aerosol-generating device 100. During the operation of the aerosol-generating device 100, ambient air entering through the pores 173 in the protective grille 172 around the charging connector 170 will converge to form a combined flow that then travels to the capsule 200. For example, the pores 173 may be in fluidic communication with the capsule-receiving cavity 130. In at least one example embodiment, air may be drawn from the pores 173 and through the capsule-receiving cavity 130. For example, air may be drawn through a capsule 200 received by the capsule-receiving cavity 130 and out of the replaceable mouthpiece 190.

The capsule 200 (for example, as illustrated in FIG. 7) may have various forms and configurations. For instance, the capsule 200 may have any of the forms and configurations as subsequently discussed in U.S. application Ser. No. 17/947,436, filed on Sep. 19, 2022, the entire contents of which are herein incorporated by reference.

In at least one example embodiment, such as best illustrated in FIG. 7, the housing 120 encases or houses an air hose 180. The air hose 180 may extend between and/or physically connect the capsule-receiving cavity 130 (via an air inlet connection 184) and the one or more air inlets or pores 173. An air channel assembly 181 may also be provided as an intermediary between the air hose 180 and the pores 173. In such an instance, the air channel assembly 181 may be configured to direct the incoming airflow (that is drawn in through the pores 173) to the air hose 180. In at least one example embodiment, the air channel assembly 181 includes an airflow restrictor configured to provide optional control over the airflow through the aerosol-generating device 100. In at least one example embodiment, one or more flow sensors 185 may be disposed within or along the air channel assembly 181 and/or along the air hose 180. In at least one example embodiment, the one or more flow sensors 185 includes a microelectromechanical system (MEMS) flow or pressure sensor or another type of sensor configure to measure air flow, such as a hot-wire anemometer. In at least one example embodiment, the one or more flow sensors 185 may include pressure sensors, such as a capacitive pressure sensor, that are configured to measure a negative pressure during a draw event. In at least one example embodiment, the air channel assembly 181 may omit the one or more sensors 185.

In at least one example embodiment, the housing 120 encloses a capsule connector 132. Additionally, in some instances, the capsule connector 132 may be mounted or otherwise secured to a printed circuit board (PCB) within the housing 120. In at least one example embodiment, the capsule connector 132 defines the capsule-receiving cavity 130. The capsule connector 132 is further described in U.S. application Ser. No. 17/947,436, filed on Sep. 19, 2022, the entire contents of which are herein incorporated by reference.

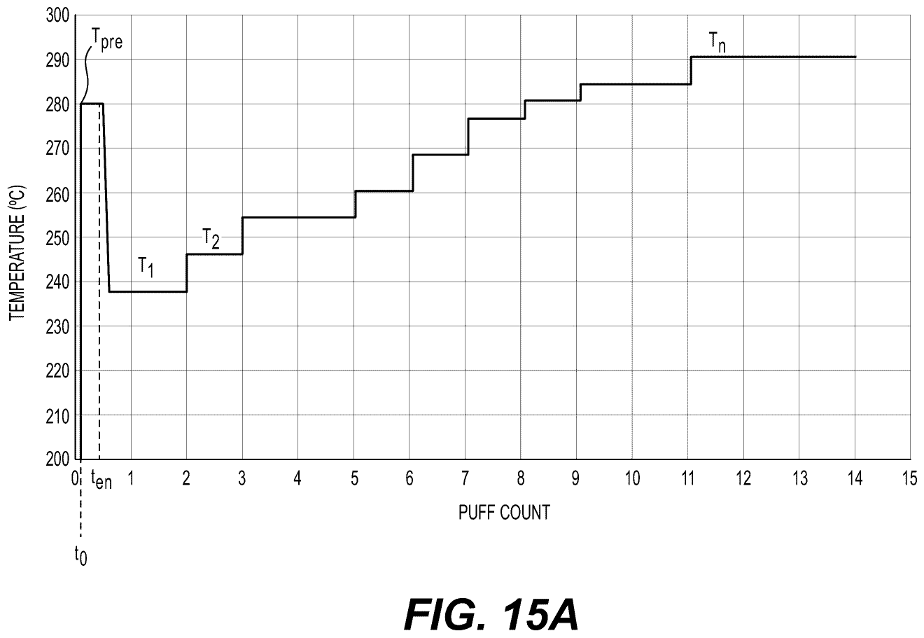

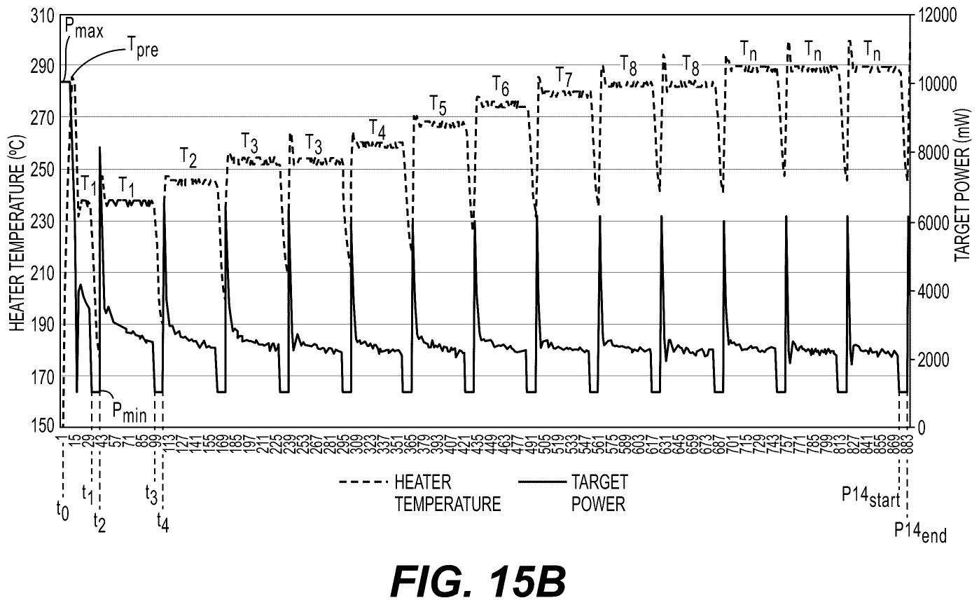

In at least one example embodiment, as shown in FIG. 6, the capsule connector 132 includes a body or housing 134 that defines the capsule-receiving cavity 130. In at least one example embodiment, the body 134 includes an air inlet connection 184. The air inlet connection 184 may be configured to be coupled to an end of the air hose 180. One or more wings or tab portions 137 and coupler-receiving openings (e.g., mounting bosses) may couple the capsule connecter 132 to the housing 120 and/or to a component within the housing. The coupler-receiving openings may be configured to receive one or more corresponding couplers of the housing 120 (such as, coupler 128 (e.g., screw) as illustrated best in FIG. 15).

In at least one example embodiments, the capsule connector 132 includes one or more electrical connectors or contacts 152A, 152B. For example, as illustrated, the capsule connector 132 may include a first electrical contact 152A and a second electrical contact 152B. As illustrated, the first electrical contact 152A may be in the form of three contact members. Similarly, the second electrical contact 152B may also be in the form of three contact members. The electrical contacts 152A, 152B are configured to apply current or other electrical signals to the capsule 200 received by the capsule-receiving cavity 130. In at least one example embodiment, the electrical contacts 152A, 152B may be in electrical communication with the power source 150 and/or control circuity 160 disposed within the housing 120. The electrical contacts 152A, 152B can be formed of copper or of a copper alloy (e.g., copper-titanium), and in at least one example embodiment, the electrical contacts 152A, 152B may have a gold plating.

The capsule 200 is loaded into the aerosol-generating device 100 by initially inserting the capsule 200 into the capsule-receiving cavity 130 defined by the capsule connector 132. In at least one example embodiment, the capsule 200 makes contact (e.g., full contact) with the electrical contacts 152A, 152B within capsule-receiving cavity 130 only upon the application of force (e.g., downward/inward force) to the capsule 200. In at least one example embodiment, a force is applied to the capsule 200 by the closure and/or latching of the lid 110. In other example embodiments, a force is applied to the capsule 200 by an adult consumer. In still other example embodiments, a force is applied by a combination of pressure applied by the adult consumer and the closure and/or latching of the lid 110. For example, in each instance, a forced is applied until a resistance is felt and/or a clicking sound is heard, which signals a complete engagement of the capsule 200 in the capsule-receiving cavity 130.

The underside of the lid 110 may include an impingement/engagement member or surface 113 configured to engage the capsule 200 when the lid 110 is pivoted to transition to a closed position. The impingement/engagement member or surface 113 of the lid 110 may include a recess (e.g., that corresponds to the size and shape of the capsule 200) and/or a resilient material to enhance an interface with the capsule 200 so as to provide the desired seal. When the capsule 200 is inserted into the capsule-receiving cavity 130, the weight of the capsule 200 itself may not be sufficient to compress the electrical contacts 152A, 152B (e.g., at least not to any significant degree). As a result, the capsule 200 may simply rest on the exposed pins of the electrical contacts 152A, 152B (e.g., contact surfaces 158A, 158B of the contact members 152′/152″) without any compression (or without any significant compression) of the electrical contacts 152A, 152B. Additionally, the weight of the lid 110 itself, when pivoted to transition to a closed position, may not compress the electrical contacts 152A, 152B to any significant degree and, instead, may simply rest on the capsule 200 in an intermediate, partially open/closed position. In such an instance, a deliberate action (e.g., downward force) to close the lid 110 will cause the impingement/engagement member or surface 113 of the lid 110 to press down onto the capsule 200 to provide the desired seal and also cause the capsule 200 to compress and, thus, fully engage electrical contacts 152A, 152B. Additionally, a full closure of the lid 110 will result in an engagement with the latch 114, which will maintain the closed position and the desired mechanical/electrical engagements involving the capsule 200 until released (e.g., via the latch release button 118). The force requirement for closing the lid 110 may help to ensure and/or improve air/aerosol sealing and to provide a more robust electrical connection, as well as improved device and thermal efficiency and battery life by reducing or eliminating early power draws and/or parasitic heating of the capsule 200.

In at least one example embodiment, as best illustrated in FIG. 5, a bottom end 166b of the capsule-receiving cavity 130 includes a capsule seal 202. When the capsule 200 is seated within the capsule-receiving cavity 130, the capsule seal 202 is configured to mate with the inlet recess of the capsule 200 (e.g., inlet recess of the capsule 200 analogous to the inlet recess 1328 of the capsule 1300). The capsule seal 202 may be configured to help ensure and/or improve air/aerosol sealing between the capsule 200 and the capsule connector 132 such that all (or substantially all) of the air received via the air inlet connection 184 is directed into the capsule 200. In at least one example embodiment, the capsule seal 202 may be a silicone seal.

When the capsule 200 is inserted into the capsule-receiving cavity 130, the end sections of the capsule 200 may initially come to rest on the electrical contacts 152A, 152B. A downward/inward force on the capsule 200 (e.g., via the closing of the lid 110) will urge the capsule 200 downward/inward so as to cause the electrical contacts 152A, 152B to compress and, thus, retract into the capsule connector 132.

As discussed herein, an aerosol-forming substrate is a material or combination of materials that may yield an aerosol. An aerosol relates to the matter generated or output by the devices disclosed, claimed, and equivalents thereof. The material may include a compound (e.g., nicotine, cannabinoid), where an aerosol including the compound is produced when the material is heated. The heating may be below the combustion temperature so as to produce an aerosol without involving a substantial pyrolysis of the aerosol-forming substrate or the substantial generation of combustion byproducts (if any). Thus, in at least one example embodiment, pyrolysis does not occur during the heating and resulting production of aerosol. In other instances, there may be some pyrolysis and combustion byproducts, but the extent may be considered relatively minor and/or merely incidental.

The aerosol-forming substrate may be a fibrous material. For instance, the fibrous material may be a botanical material. The fibrous material is configured to release a compound when heated. The compound may be a naturally occurring constituent of the fibrous material. For instance, the fibrous material may be plant material such as tobacco, and the compound released may be nicotine. The term “tobacco” includes any tobacco plant material including tobacco leaf, tobacco plug, reconstituted tobacco, compressed tobacco, shaped tobacco, or powder tobacco, and combinations thereof from one or more species of tobacco plants, such as Nicotiana rustica and Nicotiana tabacum.

In some example embodiments, the tobacco material may include material from any member of the genus Nicotiana. In addition, the tobacco material may include a blend of two or more different tobacco varieties. Examples of suitable types of tobacco materials that may be used include, but are not limited to, flue-cured tobacco, Burley tobacco, Dark tobacco, Maryland tobacco, Oriental tobacco, rare tobacco, specialty tobacco, blends thereof, and the like. The tobacco material may be provided in any suitable form, including, but not limited to, tobacco lamina, processed tobacco materials, such as volume expanded or puffed tobacco, processed tobacco stems, such as cut-rolled or cut-puffed stems, reconstituted tobacco materials, blends thereof, and the like. In some example embodiments, the tobacco material is in the form of a substantially dry tobacco mass. Furthermore, in some instances, the tobacco material may be mixed and/or combined with at least one of propylene glycol, glycerin, sub-combinations thereof, or combinations thereof.

The compound may also be a naturally occurring constituent of a medicinal plant that has a medically-accepted therapeutic effect. For instance, the medicinal plant may be a cannabis plant, and the compound may be a cannabinoid. Cannabinoids interact with receptors in the body to produce a wide range of effects. As a result, cannabinoids have been used for a variety of medicinal purposes (e.g., treatment of pain, nausea, epilepsy, psychiatric disorders). The fibrous material may include the leaf and/or flower material from one or more species of cannabis plants such as Cannabis sativa, Cannabis indica, and Cannabis ruderalis. In some instances, the fibrous material is a mixture of 60-80% (e.g., 70%) Cannabis sativa and 20-40% (e.g., 30%) Cannabis indica.

Examples of cannabinoids include tetrahydrocannabinolic acid (THCA), tetrahydrocannabinol (THC), cannabidiolic acid (CBDA), cannabidiol (CBD), cannabinol (CBN), cannabicyclol (CBL), cannabichromene (CBC), and cannabigerol (CBG). Tetrahydrocannabinolic acid (THCA) is a precursor of tetrahydrocannabinol (THC), while cannabidiolic acid (CBDA) is precursor of cannabidiol (CBD). Tetrahydrocannabinolic acid (THCA) and cannabidiolic acid (CBDA) may be converted to tetrahydrocannabinol (THC) and cannabidiol (CBD), respectively, via heating. In at least one example embodiment, heat from a heater may cause decarboxylation so as to convert the tetrahydrocannabinolic acid (THCA) in the capsule to tetrahydrocannabinol (THC), and/or to convert the cannabidiolic acid (CBDA) in the capsule to cannabidiol (CBD).

In instances where both tetrahydrocannabinolic acid (THCA) and tetrahydrocannabinol (THC) are present in the capsule, the decarboxylation and resulting conversion will cause a decrease in tetrahydrocannabinolic acid (THCA) and an increase in tetrahydrocannabinol (THC). At least 50% (e.g., at least 87%) of the tetrahydrocannabinolic acid (THCA) may be converted to tetrahydrocannabinol (THC) during the heating of the capsule. Similarly, in instances where both cannabidiolic acid (CBDA) and cannabidiol (CBD) are present in the capsule, the decarboxylation and resulting conversion will cause a decrease in cannabidiolic acid (CBDA) and an increase in cannabidiol (CBD). At least 50% (e.g., at least 87%) of the cannabidiolic acid (CBDA) may be converted to cannabidiol (CBD) during the heating of the capsule.

Furthermore, the compound may be or may additionally include a non-naturally occurring additive that is subsequently introduced into the fibrous material. In one instance, the fibrous material may include at least one of cotton, polyethylene, polyester, rayon, combinations thereof, or the like (e.g., in a form of a gauze). In another instance, the fibrous material may be a cellulose material (e.g., non-tobacco and/or non-cannabis material). In either instance, the compound introduced may include nicotine, cannabinoids, and/or flavorants. The flavorants may be from natural sources, such as plant extracts (e.g., tobacco extract, cannabis extract), and/or artificial sources. In yet another instance, when the fibrous material includes tobacco and/or cannabis, the compound may be or may additionally include one or more flavorants (e.g., menthol, mint, vanilla). Thus, the compound within the aerosol-forming substrate may include naturally occurring constituents and/or non-naturally occurring additives. In this regard, it should be understood that existing levels of the naturally occurring constituents of the aerosol-forming substrate may be increased through supplementation. For example, the existing levels of nicotine in a quantity of tobacco may be increased through supplementation with an extract containing nicotine. Similarly, the existing levels of one or more cannabinoids in a quantity of cannabis may be increased through supplementation with an extract containing such cannabinoids.

The aerosol-generating device in accordance with at least some example embodiments (such as, the aerosol-generating device 100 illustrated in FIGS. 1-5) are configured to heat a capsule (e.g., capsule 200) to generate an aerosol. In at least one example embodiment, a method of generating an aerosol may include initially loading a capsule 200 into the aerosol-generating device 100 or the aerosol-generating device 500. To load the capsule 200, the lid 110 is pivoted to the open position, and the capsule 200 is inserted into the capsule-receiving cavity 130 defined by the capsule connector 132. Next, pivoting the lid 110 to the closed position such that the lid 110 engages the latch 114 and will maintain the closed position while pressing the capsule 200 further into the capsule-receiving cavity 130 to fully seat the capsule 200.

The aerosol-generating device 100 may be activated using the consumer interface panel 143 (e.g., by pressing the power button 142) and/or upon the detection of a draw event (e.g., via the flow sensor 185). Upon activation, the control circuitry 160 is configured to instruct the power source 150 to supply an electrical current to the capsule 200 via the electrical contacts 152A, 152B in the capsule-receiving cavity 130. Specifically, as shown in FIGS. 5-6, the capsule 200 includes a heater 336 that is configured to undergo resistive heating in response to the electrical current from the power source 150 that is introduced via its end sections (which may be analogous to the first end section 1342 and the second end section 1346 of the capsule 1300). As a result of the resistive heating, the temperature of the aerosol-forming substrate within the capsule 200 will increase such that volatiles are released so as to generate an aerosol. In some example embodiments, inductive heating may be used instead of resistive heating or in combination with resistive heating. While FIGS. 5-6 illustrates the heater 336 as part of the capsule 200, example embodiments are not limited thereto. For example, the heater 336 may be part of the device 100 and external to the capsule 200, such as described in U.S. application Ser. No. 17/579,439, filed Jan. 19, 2022, the entire contents of which are herein incorporated by reference.

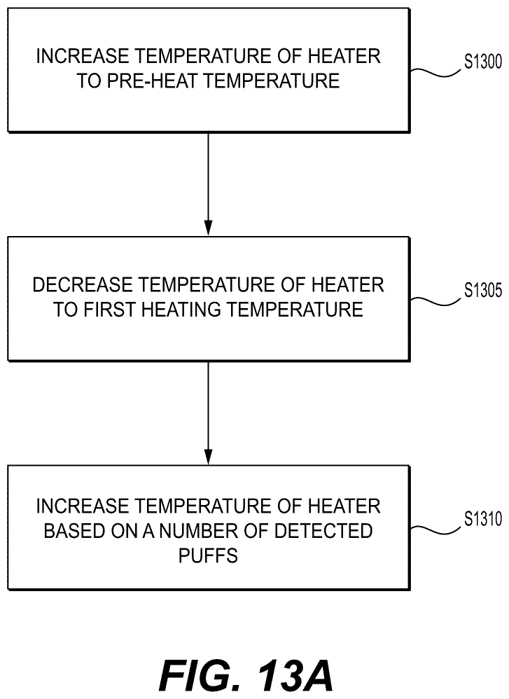

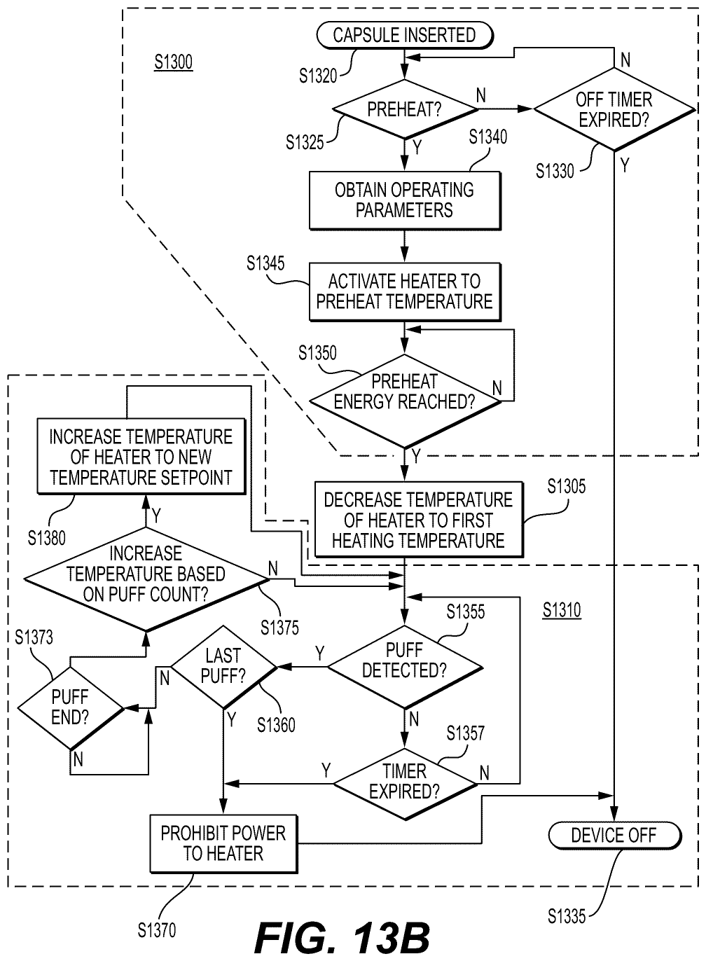

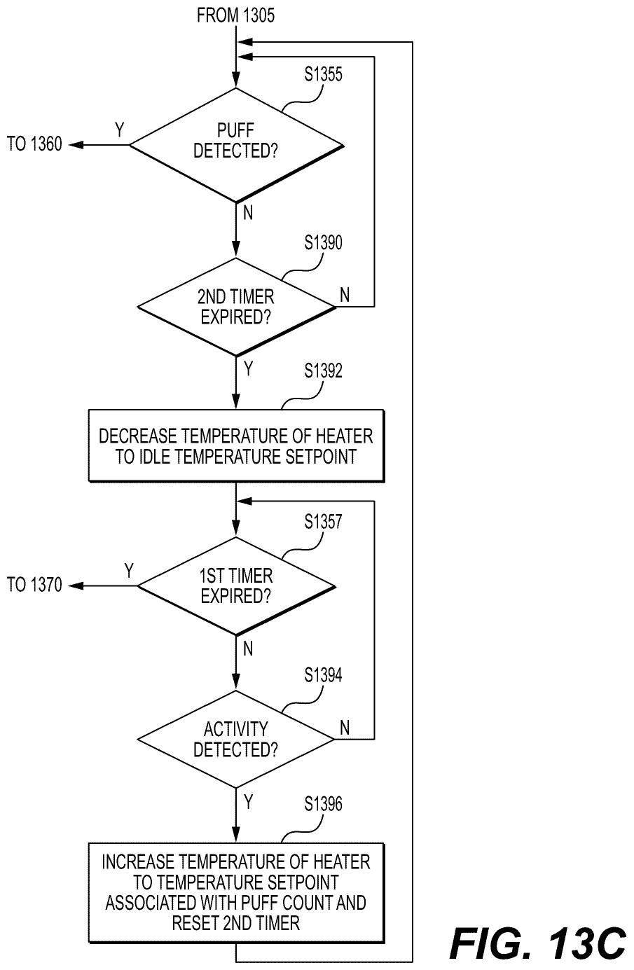

In at least one example embodiment, the heating of the aerosol-forming substrate within the capsule 200 may be below a combustion temperature of the aerosol-forming substrate so as to produce an aerosol without involving a substantial pyrolysis of the aerosol-forming substrate or the substantial generation of combustion byproducts (if any). Thus, in at least one example embodiment, pyrolysis does not occur during the heating and resulting production of aerosol. In other instances, there may be some pyrolysis and combustion byproducts, but the extent may be considered relatively minor and/or merely incidental. The method of heating/control is described below with reference to FIGS. 13A-16B.

Upon a draw or application of negative pressure to the aerosol-generating device 100 (e.g., via the mouthpiece 190), ambient air is drawn into the aerosol-generating device 100 through the pores 173 in the grille 172. Once inside, the air streams from the pores 173 converge and may pass through an air channel assembly 181 before being directed to the air hose 180. The converged airflow may be optionally detected/monitored with a flow sensor 185 within the air channel assembly 181 and/or the air hose 180. From the air hose 180, the airflow is directed to the air inlet connection 184 of the capsule connector 132. The airflow then travels through the capsule seal 202 and enters the inlet openings in the capsule 200. Inside the capsule 200, the air may flow (e.g., longitudinally) through the aerosol-forming substrate and along the plane of the heater so as to entrain the volatiles released by the aerosol-forming substrate, which results in an aerosol. Finally, the resulting aerosol passes through the outlet openings in the capsule 200 before exiting the aerosol-generating device 100 (e.g., via the outlets 196 in the mouthpiece 190).

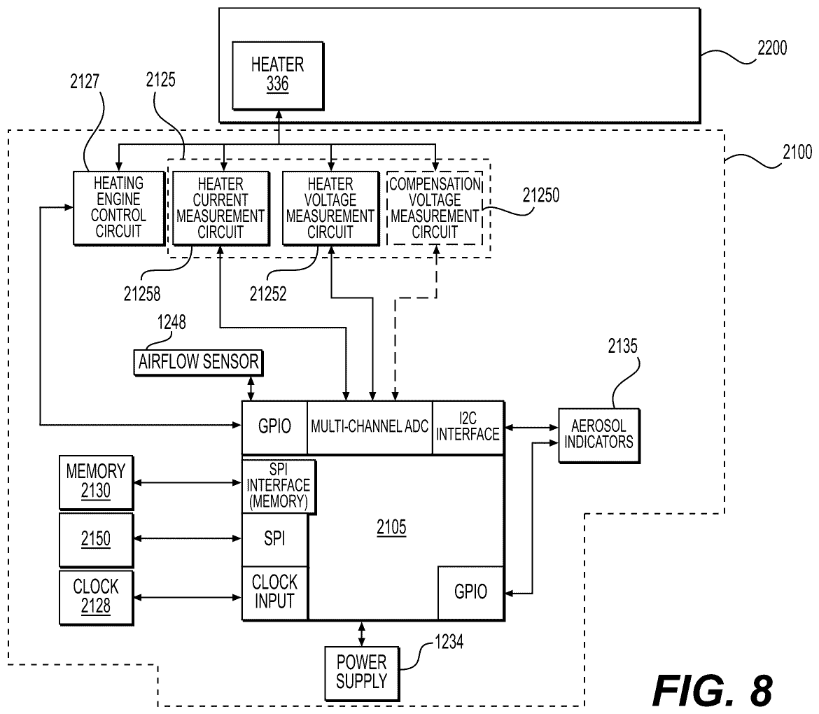

FIG. 8 illustrates electrical systems of an aerosol-generating device and a capsule according to one or more example embodiments.

Referring to FIG. 8, the electrical systems include an aerosol-generating device electrical system 2100 and a capsule electrical system 2200. The aerosol-generating device electrical system 2100 may be included in the aerosol-generating device 100, and the capsule electrical system 2200 may be included in the capsule 200.

In the example embodiment shown in FIG. 8, the capsule electrical system 2200 includes the heater 336.

The capsule electrical system 2200 may further include a body electrical/data interface (not shown) for transferring power and/or data between the aerosol-generating device 100 and the capsule 200. According to at least one example embodiment, the electrical contacts shown in FIG. 6, for example, may serve as the body electrical interface, but example embodiments are not limited thereto.

The aerosol-generating device electrical system 2100 includes a controller 2105, a power supply 1234, device sensors or measurement circuits 2125, a heating engine control circuit 2127, aerosol indicators 2135, on-product controls 2150 (e.g., buttons shown in FIG. 1), a memory 2130, and a clock circuit 2128. In some example embodiments, the controller 2105, the power supply 1234, device sensors or measurement circuits 2125, the heating engine control circuit 2127, the memory 2130, and the clock circuit 2128 are on the same PCB (e.g., the main PCB 1246). The aerosol-generating device electrical system 2100 may further include a capsule electrical/data interface (not shown) for transferring power and/or data between the aerosol-generating device 100 and the capsule 200.

The power supply 1234 may be an internal power supply to supply power to the aerosol-generating device 100 and the capsule 200. The supply of power from the power supply 1234 may be controlled by the controller 2105 through power control circuitry (not shown). The power control circuitry may include one or more switches or transistors to regulate power output from the power supply 1234. The power supply 1234 may be a Lithium-ion battery or a variant thereof (e.g., a Lithium-ion polymer battery).

The controller 2105 may be configured to control overall operation of the aerosol-generating device 100. According to at least some example embodiments, the controller 2105 may include processing circuitry such as hardware including logic circuits; a hardware/software combination such as a processor executing software; or a combination thereof. For example, the processing circuitry more specifically may include, but is not limited to, a central processing unit (CPU), an arithmetic logic unit (ALU), a digital signal processor, a microcomputer, a field programmable gate array (FPGA), a System-on-Chip (SoC), a programmable logic unit, a microprocessor, application-specific integrated circuit (ASIC), etc.

In the example embodiment shown in FIG. 8, the controller 2105 is illustrated as a microcontroller including: input/output (I/O) interfaces, such as general purpose input/outputs (GPIOs), inter-integrated circuit (I²C) interfaces, serial peripheral interface bus (SPI) interfaces, or the like; a multichannel analog-to-digital converter (ADC); and a clock input terminal. However, example embodiments should not be limited to this example. In at least one example implementation, the controller 2105 may be a microprocessor.

The memory 2130 is illustrated as being external to the controller 2105, in some example embodiments the memory 2130 may be on board the controller 2105.

The controller 2105 is communicatively coupled to the device sensors 2125, the heating engine control circuit 2127, aerosol indicators 2135, the memory 2130, the on-product controls 2150, the clock circuit 2128 and the power supply 1234.

The heating engine control circuit 2127 is connected to the controller 2105 via a GPIO (General Purpose Input/Output) pin. The memory 2130 is connected to the controller 2105 via a SPI (Serial Peripheral Interface) pin. The clock circuit 2128 is connected to a clock input pin of the controller 2105. The aerosol indicators 2135 are connected to the controller 2105 via an I²C (Inter-Integrated Circuit) interface pin and a SPI/GPIO pin. The device sensors 2125 are connected to the controller 2105 through respective pins of the multi-channel ADC.

The clock circuit 2128 may be a timing mechanism, such as an oscillator circuit, to enable the controller 2105 to track idle time, preheat length, aerosol-generating (draw) length, a combination of idle time and aerosol-generating (draw) length, a power-use time to determine a hot capsule alert (e.g., 30 s after instance has ended) or the like, of the aerosol-generating device 10. The clock circuit 2128 may also include a dedicated external clock crystal configured to generate the system clock for the aerosol-generating device 10.

The memory 2130 may be a non-volatile memory storing operational parameters and computer readable instructions for the controller 2105 to perform the algorithms described herein. In one example, the memory 2130 may be an electrically erasable programmable read-only memory (EEPROM), such as a flash memory or the like.

Still referring to FIG. 8, the device sensors 2125 may include a plurality of sensor or measurement circuits configured to provide signals indicative of sensor or measurement information to the controller 2105. In the example shown in FIG. 8, the device sensors 2125 include a heater current measurement circuit 21258, a heater voltage measurement circuit 21252, and a compensation voltage measurement circuit 21250. The electrical systems of FIG. 8 may further include the sensors discussed with reference to FIGS. 1-7.

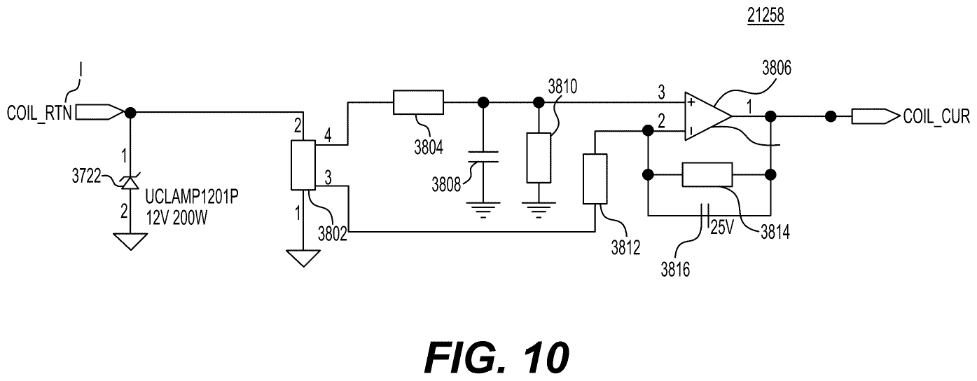

The heater current measurement circuit 21258 may be configured to output (e.g., voltage) signals indicative of the current through the heater 336. An example embodiment of the heater current measurement circuit 21258 will be discussed in more detail later with regard to FIG. 10.

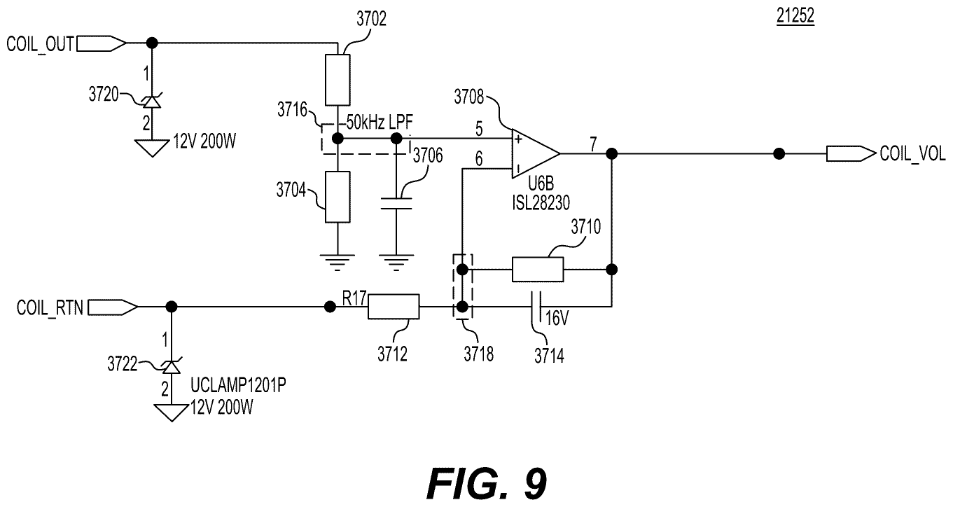

The heater voltage measurement circuit 21252 may be configured to output (e.g., voltage) signals indicative of the voltage across the heater 336. An example embodiment of the heater voltage measurement circuit 21252 will be discussed in more detail later with regard to FIG. 9.

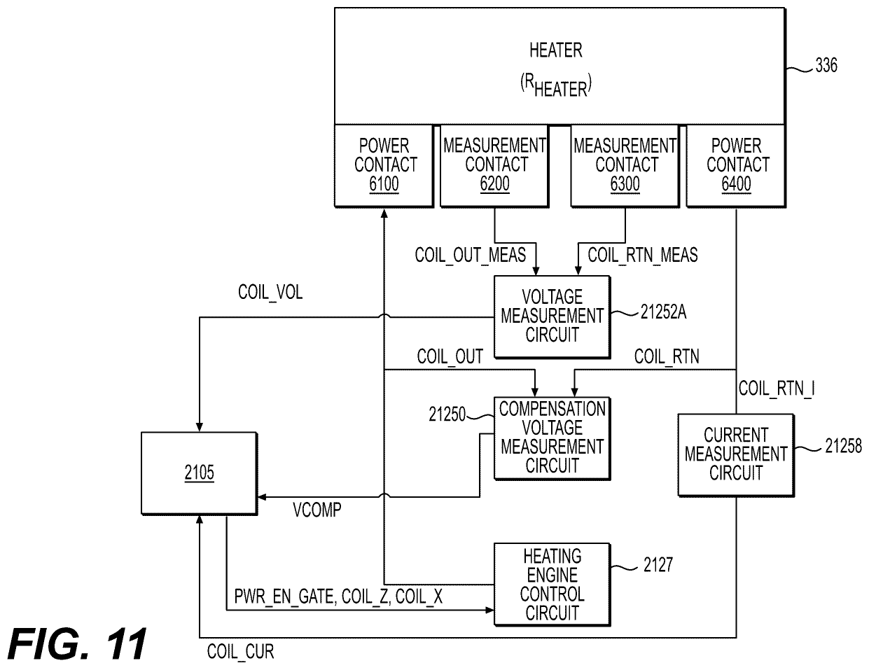

The compensation voltage measurement circuit 21250 may be configured to output (e.g., voltage) signals indicative of the resistance of electrical power interface (e.g., electrical connector) between the capsule 200 and the aerosol-generating device 100. In some example embodiments, the compensation voltage measurement circuit 21250 may provide compensation voltage measurement signals to the controller 2105. Example embodiments of the compensation voltage measurement circuit 21250 will be discussed in more detail later with regard to FIG. 11 and are described in U.S. application Ser. No. 17/151,375, filed Jan. 18, 2021, the entire contents of which are herein incorporated by reference.

As discussed above, the compensation voltage measurement circuit 21250, the heater current measurement circuit 21258 and the heater voltage measurement circuit 21252 are connected to the controller 2105 via pins of the multi-channel ADC. To measure characteristics and/or parameters of the aerosol-generating device 100 and the capsule 200 (e.g., voltage, current, resistance, temperature, or the like, of the heater 336), the multi-channel ADC at the controller 2105 may sample the output signals from the device sensors 2125 at a sampling rate appropriate for the given characteristic and/or parameter being measured by the respective device sensor.

The aerosol-generating device electrical system 2100 may include the sensor 1248 to measure airflow through the aerosol-generating device 100. In at least one example embodiment, the sensor may be a microelectromechanical system (MEMS) flow or pressure sensor or another type of sensor configured to measure air flow such as a hot-wire anemometer. In an example embodiment, the output of the sensor to measure airflow to the controller 2105 is instantaneous measurement of flow (in ml/s or cm³/s) via a digital interface or SPI. In other example embodiments, the sensor may be a hot-wire anemometer, a digital MEMS sensor or other known sensors. The flow sensor may be operated as a puff sensor by detecting a draw when the flow value is greater than or equal to 1 mL/s, and terminating a draw when the flow value subsequently drops to 0 mL/s. In an example embodiment, the sensor 1248 may be a MEMS flow sensor based differential pressure sensor with the differential pressure (in Pascals) converted to an instantaneous flow reading (in mL/s) using a curve fitting calibration function or a Look Up Table (of flow values for each differential pressure reading). In another example embodiment, the flow sensor may be a capacitive pressure drop sensor.

The heating engine control circuit 2127 is connected to the controller 2105 via a GPIO pin. The heating engine control circuit 2127 is configured to control (enable and/or disable) the heater 336 of the aerosol-generating device 10 by controlling power to the heater 336.

The controller 2105 may control the aerosol indicators 2135 to indicate statuses and/or operations of the aerosol-generating device 10 to an adult operator. The aerosol indicators 2135 may be at least partially implemented via a light guide and may include a power indicator (e.g., LED) that may be activated when the controller 2105 senses a button pressed by the adult operator. The aerosol indicators 2135 may also include a vibrator, speaker, or other feedback mechanisms, and may indicate a current state of an adult operator-controlled aerosol generating parameter (e.g., aerosol volume).

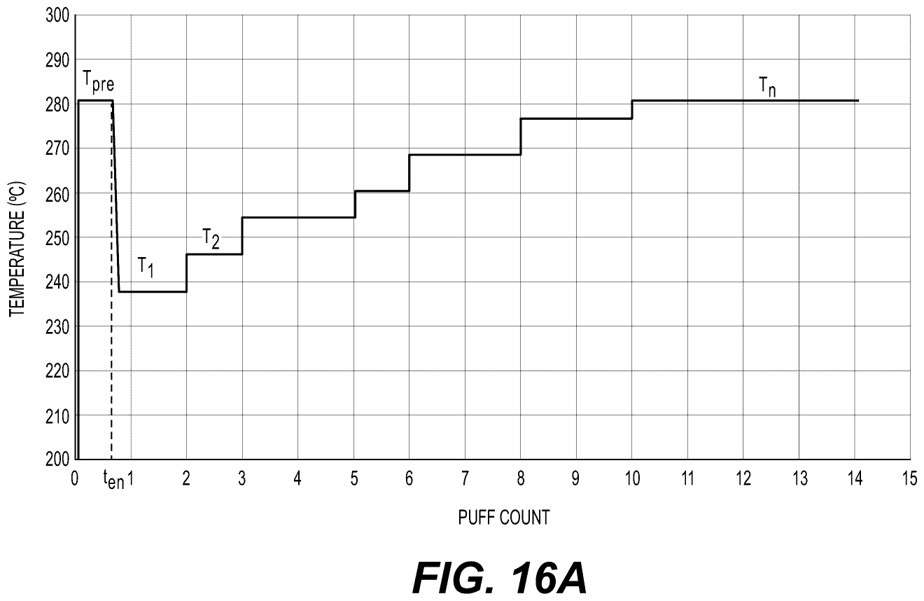

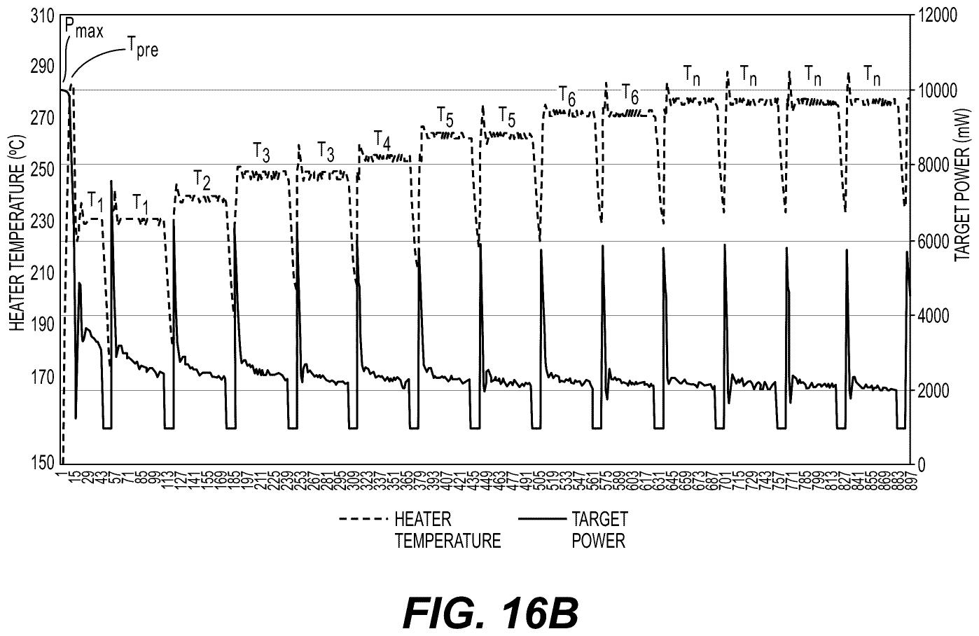

Still referring to FIG. 8, the controller 2105 may control power to the heater 336 to heat the aerosol-forming substrate in accordance with a heating profile (e.g., heating based on volume, temperature, flavor, or the like). The heating profile may be determined based on empirical data and may be stored in the memory 2130 of the aerosol-generating device 100. Example embodiments of heating profiles are described with reference to FIGS. 15A-16B.

FIG. 9 illustrates an example embodiment of the heater voltage measurement circuit 21252.

Referring to FIG. 9, the heater voltage measurement circuit 21252 includes a resistor 3702 and a resistor 3704 connected in a voltage divider configuration between a terminal configured to receive an input voltage signal COIL_OUT and ground. The resistances of the resistor 3702 and the resistor 3704 may be 8.2 kiloohms and 3.3 kiloohms, respectively. The input voltage signal COIL_OUT is the voltage input to (voltage at an input terminal of) the heater 336. A node N3716 between the resistor 3702 and the resistor 3704 is coupled to a positive input of an operational amplifier (Op-Amp) 3708. A capacitor 3706 is connected between the node N3716 and ground to form a low-pass filter circuit (an R/C filter) to stabilize the voltage input to the positive input of the Op-Amp 3708. The capacitance of the capacitor 3706 may be 18 nanofarads, for example. The filter circuit may also reduce inaccuracy due to switching noise induced by PWM signals used to energize the heater 336, and have the same phase response/group delay for both current and voltage.

The heater voltage measurement circuit 21252 further includes resistors 3710 and 3712 and a capacitor 3714. The resistor 3712 is connected between node N3718 and a terminal configured to receive an output voltage signal COIL_RTN and may have a resistance of 8.2 kiloohms, for example. The output voltage signal COIL_RTN is the voltage output from (voltage at an output terminal of) the heater 336.

Resistor 3710 and capacitor 3714 are connected in parallel between a node N3718 and an output of the Op-Amp 3708. The resistor 3710 may have a resistance of 3.3 kiloohms and the capacitor 3714 may have a capacitance of 18 nanofarads, for example. A negative input of the Op-Amp 3708 is also connected to node N3718. The resistors 3710 and 3712 and the capacitor 3714 are connected in a low-pass filter circuit configuration.

The heater voltage measurement circuit 21252 utilizes the Op-Amp 3708 to measure the voltage differential between the input voltage signal COIL_OUT and the output voltage signal COIL_RTN, and output a scaled heater voltage measurement signal COIL_VOL that represents the voltage across the heater 336. The heater voltage measurement circuit 21252 outputs the scaled heater voltage measurement signal COIL_VOL to an ADC pin of the controller 2105 for digital sampling and measurement by the controller 2105.

The gain of the Op-Amp 3708 may be set based on the surrounding passive electrical elements (e.g., resistors and capacitors) to improve the dynamic range of the voltage measurement. In one example, the dynamic range of the Op-Amp 3708 may be achieved by scaling the voltage so that the maximum voltage output matches the maximum input range of the ADC (e.g., about 2.5V). In at least one example embodiment, the scaling may be about 402 mV per V, and thus, the heater voltage measurement circuit 21252 may measure up to about 2.5V/0.402V=6.22V.

The voltage signals COIL_OUT and COIL_RTN are clamped by diodes 3720 and 3722, respectively, to reduce risk of damage due to electrostatic discharge (ESD) events.

In some example embodiments, four wire/Kelvin measurement may be used and the voltage signals COIL_OUT and COIL_RTN may be measured at measurement contact points (also referred to as voltage sensing connections (as opposed to main power contacts)) to take into account the contact and bulk resistances of an electrical power interface (e.g., electrical connector) between the heater 336 and the aerosol-generating device 100.

FIG. 10 illustrates an example embodiment of the heater current measurement circuit 21258 shown in FIG. 8.

Referring to FIG. 10, an output current signal COIL_RTN_I is input to a four terminal (4T) measurement resistor 3802 connected to ground. The differential voltage across the four terminal measurement resistor 3802 is scaled by an Op-Amp 3806, which outputs a heater current measurement signal COIL_CUR indicative of the current through the heater 336. The heater current measurement signal COIL_CUR is output to an ADC pin of the controller 2105 for digital sampling and measurement of the current through the heater 336 at the controller 2105.

In the example embodiment shown in FIG. 10, the four terminal measurement resistor 3802 may be used to reduce error in the current measurement using a four wire/Kelvin current measurement technique. In this example, separation of the current measurement path from the voltage measurement path may reduce noise on the voltage measurement path.

The gain of the Op-Amp 3806 may be set to improve the dynamic range of the measurement. In this example, the scaling of the Op-Amp 3806 may be about 0.820 V/A, and thus, the heater current measurement circuit 21258 may measure up to about 2.5 V/(0.820 V/A)=3.05 A.

Referring to FIG. 10 in more detail, a first terminal of the four terminal measurement resistor 3802 is connected to a terminal of the heater 336 to receive the output current signal COIL_RTN_I. A second terminal of the four terminal measurement resistor 3802 is connected to ground. A third terminal of the four terminal measurement resistor 3802 is connected to a low-pass filter circuit (R/C filter) including resistor 3804, capacitor 3808 and resistor 3810. The resistance of the resistor 3804 may be 100 ohms, the resistance of the resistor 3810 may be 8.2kiloohms and the capacitance of the capacitor 3808 may be 3.3. nanofarads, for example.

The output of the low-pass filter circuit is connected to a positive input of the Op-Amp 3806. The low-pass filter circuit may reduce inaccuracy due to switching noise induced by the PWM signals applied to energize the heater 336, and may also have the same phase response/group delay for both current and voltage.

The heater current measurement circuit 21258 further includes resistors 3812 and 3814 and a capacitor 3816. The resistors 3812 and 3814 and the capacitor 3816 are connected to the fourth terminal of the four terminal measurement resistor 3802, a negative input of the Op-Amp 3806 and an output of the Op-Amp 3806 in a low-pass filter circuit configuration, wherein the output of the low-pass filter circuit is connected to the negative input of the Op-Amp 3806. The resistors 3812 and 3814 may have resistances of 100 ohms and 8.2 kiloohms, respectively, and the capacitor 3816 may have a capacitance of 3.3. nanofarads, for example.

The Op-Amp 3806 outputs a differential voltage as the heater current measurement signal COIL_CUR to an ADC pin of the controller 2105 for sampling and measurement of the current through the heater 336 by the controller 2105.

According to at least this example embodiment, the configuration of the heater current measurement circuit 21258 is similar to the configuration of the heater voltage measurement circuit 21252, except that the low-pass filter circuit including resistors 3804 and 3810 and the capacitor 3808 is connected to a terminal of the four terminal measurement resistor 3802 and the low-pass filter circuit including the resistors 3812 and 3814 and the capacitor 3816 is connected to another terminal of the four terminal measurement resistor 3802.

The controller 2105 may average multiple samples (e.g., of voltage) over a time window (e.g., about 1 ms) corresponding to the ‘tick’ time (iteration time of a control loop) used in the aerosol-generating device 100, and convert the average to a mathematical representation of the voltage and current across the heater 336 through application of a scaling value. The scaling value may be determined based on the gain settings implemented at the respective Op-Amps, which may be specific to the hardware of the aerosol-generating device 100.

The controller 2105 may filter the converted voltage and current measurements using, for example, a three tap moving average filter to attenuate measurement noise. The controller 2105 may then use the filtered measurements to calculate: resistance R_{HEATER} of the heater 336 (R_{HEATER}=COIL_VOL/COIL_CUR), power P_{HEATER} applied to the heater 336 (P_{HEATER}=COIL_VOL*COIL_CUR) or the like.

According to one or more example embodiments, the gain settings of the passive elements of the circuits shown in FIGS. 9 and/or 10 may be adjusted to match the output signal range to the input range of the controller 2105.

FIG. 11 illustrates electrical systems of an aerosol-generating device including a separate compensation voltage measurement circuit according to one or more example embodiments.

As shown in FIG. 11, a contact interface between the heater 336 and the aerosol-generating device electrical system 2100 includes a four wire/Kelvin arrangement having an input power contact 6100, an input measurement contact 6200, an output measurement contact 6300 and an output power contact 6400.

A voltage measurement circuit 21252A receives a measurement voltage COIL_OUT_MEAS at the input measurement contact 6200 and an output measurement voltage COIL_RTN_MEAS at the output measurement contact 6300. The voltage measurement circuit 21252A is the same circuit as the voltage measurement circuit 21252 illustrated in FIG. 9 and outputs the scaled heater voltage measurement signal COIL_VOL. While in FIG. 9 COIL_OUT and COIL_RTN are illustrated, it should be understood that in example embodiments without a separate compensation voltage measurement circuit, the voltage measurement circuit 21252 may receive voltages at the input and output measurement contacts 6200, 6300 instead of the input and output power contacts 6100, 6400.

The systems shown in FIG. 11 further include the compensation voltage measurement circuit 21250. The compensation voltage measurement circuit 21250 is the same as the voltage measurement circuit 21252A except the compensation voltage measurement circuit 21250 receives the voltage COIL_OUT at the input power contact 6100 and receives the voltage COIL_RTN at the output power contact 6400 and outputs a compensation voltage measurement signal VCOMP.

The current measurement circuit 21258 receives the output current signal COIL_RTN_I at the output power contact 6400 and outputs the heater current measurement signal COIL_CUR.

The compensation voltage measurement signal VCOMP may be used to adjust a target power for a heater as described in in U.S. application Ser. No. 17/151,375, filed Jan. 18, 2021, the entire contents of which are herein incorporated by reference.

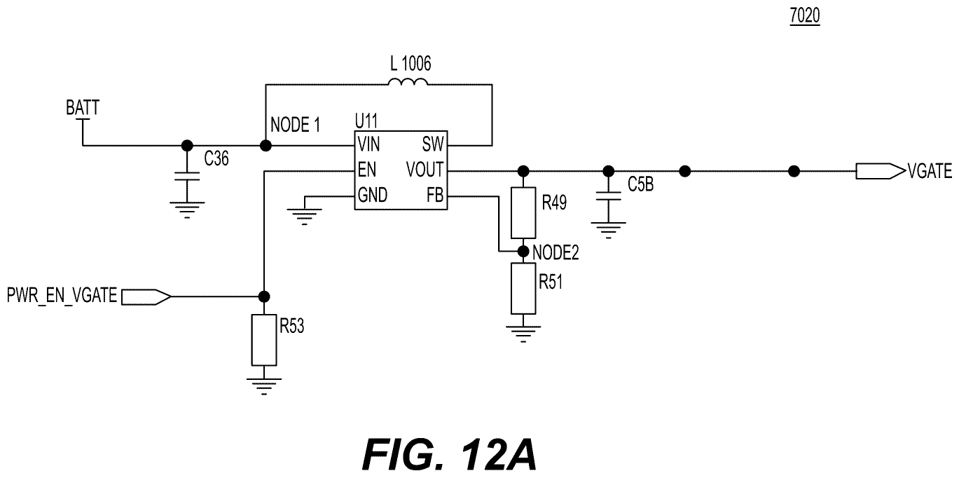

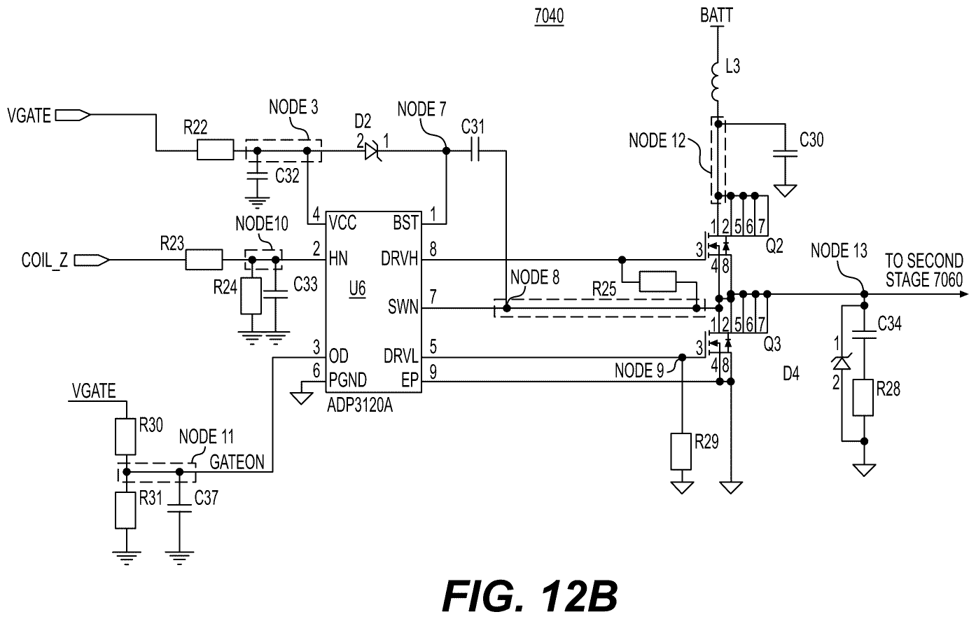

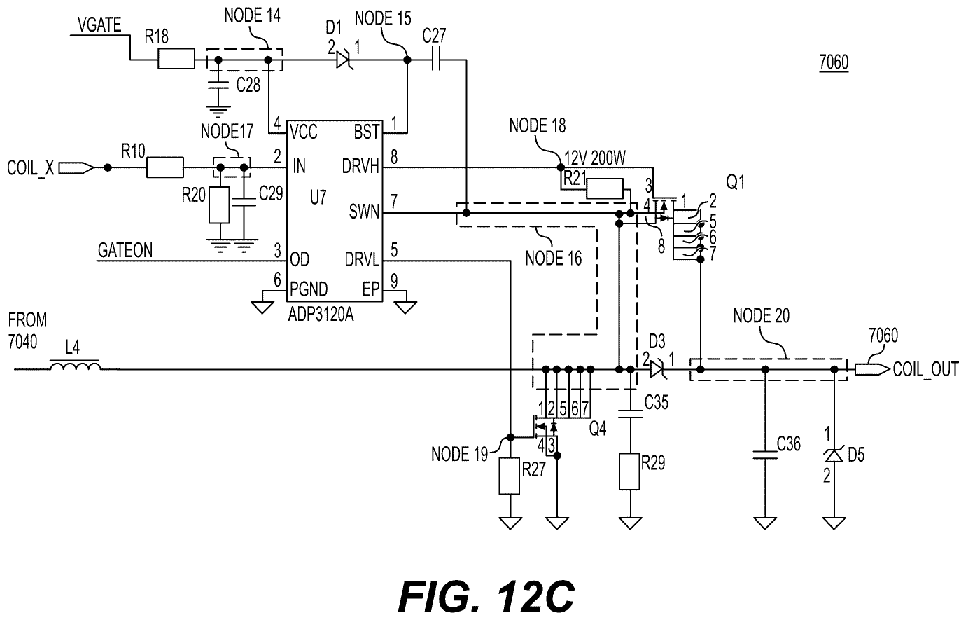

FIGS. 12A-12C is a circuit diagram illustrating a heating engine control circuit according to example embodiments. The heating engine control circuit shown in FIGS. 12A-12C is an example of the heating engine control circuit 2127 shown in FIG. 8.

The heating engine control circuit includes a boost converter circuit 7020 (FIG. 12A), a first stage 7040 (FIG. 12B) and a second stage 7060 (FIG. 12C).

The boost converter circuit 7020 is configured to create a voltage signal VGATE (e.g., 9V supply) (also referred to as a power signal or input voltage signal) from a voltage source BATT to power the first stage 7040 based on a first power enable signal PWR_EN_VGATE (also referred to as a shutdown signal). The controller may generate the first power enable signal PWR_EN_VGATE to have a logic high level when the aerosol-generating device is ready to be used. In other words, the first power enable signal PWR_EN_VGATE has a logic high level when at least the controller detects that a capsule is properly connected to the aerosol-generating device. In other example embodiments, the first power enable signal PWR_EN_VGATE has a logic high level when the controller detects that a capsule is properly connected to the aerosol-generating device and the controller detects an action such as a button being pressed.

The first stage 7040 utilizes the input voltage signal VGATE from the boost converter circuit 7020 to drive the heating engine control circuit 2127. The first stage 7040 and the second stage 7060 form a buck-boost converter circuit.

In the example embodiment shown in FIG. 12A, the boost converter circuit 7020 generates the input voltage signal VGATE only if the first enable signal PWR_EN_VGATE is asserted (present). The controller 2105 may VGATE to cut power to the first stage 7040 by de-asserting (stopping or terminating) the first enable signal PWR_EN_VGATE. The first enable signal PWR_EN_VGATE may serve as a device state power signal for performing an aerosol-generating-off operation at the device 1000. In this example, the controller 2105 may perform an aerosol-generating-off operation by de-asserting the first enable signal PWR_EN_VGATE, thereby disabling power to the first stage 7040, the second stage 7060 and the heater 336. The controller 2105 may then enable aerosol-generating at the device 1000 by again asserting the first enable signal PWR_EN_VGATE to the boost converter circuit 7020.

The controller 2105 may generate the first enable signal PWR_EN_VGATE at a logic level such that boost converter circuit 7020 outputs the input voltage signal VGATE having a high level (at or approximately 9V) to enable power to the first stage 7040 and the heater 336 in response to aerosol-generating conditions at the device 1000. The controller 2105 may generate the first enable signal PWR_EN_VGATE at another logic level such that boost converter circuit 7020 outputs the input voltage signal VGATE having a low level (at or approximately 0V) to disable power to the first stage 7040 and the heater 336, thereby performing a heater-off operation.

Referring in more detail to the boost converter circuit 7020 in FIG. 12A, a capacitor C36 is connected between the voltage source BATT and ground. The capacitor C36 may have a capacitance of 10 microfarads.

A first terminal of inductor L1006 is connected to node Nodel between the voltage source BATT and the capacitor C36. The inductor L1006 serves as the main storage element of the boost converter circuit 7020. The inductor L1006 may have an inductance of 10 microhenrys.

Node 1 is connected to a voltage input pin A1 a boost converter chip U11. In some example embodiments, the boost converter chip may be a TPS61046.

A second terminal of the inductor L1006 is connected to a switch pin SW of the boost converter chip U11. An enable pin EN of the booster converter chip U11 is configured to receive the first enable signal PWR_EN_VGATE from the controller 2105.

In the example shown in FIG. 12A, the boost converter chip Ull serves as the main switching element of the boost converter circuit 7020.

A resistor R53 is connected between the enable pin EN of the booster converter chip U11 and ground to act as a pull-down resistor to ensure that operation of the heater 336 is prevented when the first enable signal PWR_EN_GATE is in an indeterminate state. The resistor R53 may have a resistance of 100 kiloohms in some example embodiments.

A voltage output pin VOUT of the boost converter chip U11 is connected to a first terminal of a resistor R49 and first terminal of a capacitor C58. A second terminal of the capacitor C58 is connected to ground. A voltage output by the voltage output pin VOUT is the input voltage signal VGATE.

A second terminal of the resistor R49 and a first terminal of a resistor R51 are connected at a second node Node2. The second node Node2 is connected to a feedback pin FB of the booster converter chip U11. The booster converter chip U11 is configured to produce the input voltage signal VGATE at about 9V using the ratio of the resistance of the resistor R49 to the resistance of the resistor R51. In some example embodiments, the resistor R49 may have a resistance of 680 kiloohms and the resistor R51 may have a resistance of 66.5 kiloohms.

The capacitors C36 and C58 operate as smoothing capacitors and may have capacitances of 10 microfarads and 4.7 microfarads, respectively. The inductor L1006 may have an inductance selected based on a desired output voltage (e.g., 9V).

Referring now to FIG. 12B, the first stage 7040 receives the input voltage signal VGATE and a second enable signal COIL_Z. The second enable signal is a pulse-width-modulation (PWM) signal and is an input to the first stage 7040.

The first stage 7040 includes, among other things, an integrated gate driver U6 configured to convert low-current signal(s) from the controller 2105 to high-current signals for controlling switching of transistors of the first stage 7040. The integrated gate driver U6 is also configured to translate voltage levels from the controller 2105 to voltage levels required by the transistors of the first stage 7040. In the example embodiment shown in FIG. 12B, the integrated gate driver U6 is a half-bridge driver. However, example embodiments should not be limited to this example.

In more detail, the input voltage signal VGATE from the boost converter circuit 7020 is input to the first stage 7040 through a filter circuit including a resistor R22 and a capacitor C32. The resistor R22 may have a resistance of 10 ohms and the capacitor C32 may have a capacitance of 1 microfarad.

The filter circuit including the resistor R22 and the capacitor C32 is connected to the VCC pin (pin 4) of the integrated gate driver U6 and the anode of Zener diode D2 at node Node3. The second terminal of the capacitor C32 is connected to ground. The anode of the Zener diode D2 is connected to a first terminal of capacitor C32 and a boost pin BST (pin 1) of the integrated gate driver U6 at node Node7. A second terminal of the capacitor C31 is connected to the switching node pin SWN (pin 7) of the integrated gate driver U6 and between transistors Q2 and Q3 at node Node8. In the example embodiment shown in FIG. 12B, the Zener diode D2 and the capacitor C31 form part of a boot-strap charge-pump circuit connected between the input voltage pin VCC and the boost pin BST of the integrated gate driver U6. Because the capacitor C31 is connected to the input voltage signal VGATE from the boost converter circuit 7020, the capacitor C31 charges to a voltage almost equal to the input voltage signal VGATE through the diode D2. The capacitor C31 may have a capacitance of 220 nanofarads.

Still referring to FIG. 12B, a resistor R25 is connected between the high side gate driver pin DRVH (pin 8) and the switching node pin SWN (pin 7). A first terminal of a resistor R29 is connected to the low side gate driver pin DRVL at a node Node9. A second terminal of the resistor R29 is connected to ground.

A resistor R23 and a capacitor C33 form a filter circuit connected to the input pin IN (pin 2) of the integrated gate driver U6. The filter circuit is configured to remove high frequency noise from the second heater enable signal COIL_Z input to the input pin IN. The second heater enable signal COIL_Z is a PWM signal from the controller 2105. Thus, the filter circuit is designed to filter out high frequency components of a PWM square wave pulse train, slightly reduces the rise and fall times on the square wave edges so that transistors are turned on and off gradually.

A resistor R24 is connected to the filter circuit and the input pin IN at node Node10. The resistor R24 is used as a pull-down resistor, such that if the second heater enable signal COIL_Z is floating (or indeterminate), then the input pin IN of the integrated gate driver U6 is held at a logic low level to prevent activation of the heater 336.

A resistor R30 and a capacitor C37 form a filter circuit connected to a pin OD (pin 3) of the integrated gate driver U6. The filter circuit is configured to remove high frequency noise from the input voltage signal VGATE input to the pin OD.

A resistor R31 is connected to the filter circuit and the pin OD at node Nodell. The resistor R31 is used as a pull-down resistor, such that if the input voltage signal VGATE is floating (or indeterminate), then the pin OD of the integrated gate driver U6 is held at a logic low level to prevent activation of the heater 336. The signal output by the filter circuit formed by the resistor R30 and the capacitor C37 is referred to as filtered signal GATEON. R30 and R31 are also a divider circuit such that the signal VGATE is divided down to ˜2.5V for a transistor driver chip input.

The transistors Q2 and Q3 field-effect transistors (FETs) connected in series between the voltage source BATT and ground. In addition, a first terminal of an inductor L3 is connected to the voltage source BATT. A second terminal of the inductor L3 is connected to a first terminal of a capacitor C30 and to a drain of the transistor Q2 at a node Node12. A second terminal of the capacitor C30 is connected to ground. The inductor L3 and the capacitor C30 form a filter to reduce and/or prevent transient spikes from the voltage source BATT.

The gate of the transistor Q3 is connected to the low side gate driver pin DRVL (pin 5) of the integrated gate driver U6, the drain of the transistor Q3 is connected to the switching node pin SWN (pin 7) of the integrated gate driver U6 at node Node8, and the source of the transistor Q3 is connected to ground GND. When the low side gate drive signal output from the low side gate driver pin DRVL is high, the transistor Q3 is in a low impedance state (ON), thereby connecting the node Node8 to ground.

As mentioned above, because the capacitor C31 is connected to the input voltage signal VGATE from the boost converter circuit 7020, the capacitor C31 charges to a voltage equal or substantially equal to the input voltage signal VGATE through the diode D2.

When the low side gate drive signal output from the low side gate driver pin DRVL is low, the transistor Q3 switches to the high impedance state (OFF), and the high side gate driver pin DRVH (pin 8) is connected internally to the boost pin BST within the integrated gate driver U6. As a result, transistor Q2 is in a low impedance state (ON), thereby connecting the switching node SWN to the voltage source BATT to pull the switching node SWN (Node 8) to the voltage of the voltage source BATT.

In this case, the node Node7 is raised to a bootstrap voltage V(BST)≈V(VGATE)+V(BATT), which allows the gate-source voltage of the transistor Q2 to be the same or substantially the same as the voltage of the input voltage signal VGATE (e.g., V(VGATE)) regardless (or independent) of the voltage from the voltage source BATT. The circuit arrangement ensures that the BST voltage is not changed as the voltage of the voltage source drops, i.e., the transistors are efficiently switched even as the voltage of the voltage source BATT changes.

As a result, the switching node SWN (Node 8) provides a high current switched signal that may be used to generate a voltage output to the second stage 7060 (and a voltage output to the heater 336) that has a maximum value equal to the battery voltage source BATT, but is otherwise substantially independent of the voltage output from the battery voltage source BATT.

A first terminal of a capacitor C34 and an anode of a Zener diode D4 are connected to an output terminal to the second stage 7060 at a node Node13. The capacitor C34 and a resistor R28 are connected in series. A second terminal of the capacitor C34 and a first terminal of the resistor R28 are connected. A cathode of the Zener diode D4 and a second terminal of the resistor R28 are connected to ground.

The capacitor C34, the Zener diode D4 and the resistor R28 form a back EMF (electric and magnetic fields) prevention circuit that prevents energy from an inductor L4 (shown in FIG. 7C) from flowing back into the first stage 7040.

The resistor R25 is connected between the gate of the transistor Q2 and the drain of the transistor Q3. The resistor R25 serves as a pull-down resistor to ensure that the transistor Q2 switches to a high impedance more reliably.

The output of the first stage 7040 is substantially independent of the voltage of the voltage source and is less than or equal to the voltage of the voltage source. When the second heater enable signal COIL_Zis at 100% PWM, the transistor Q2 is always activated, and the output of the first stage 7040 is the voltage of the voltage source or substantially the voltage of the voltage source.

FIG. 12C illustrates the second stage 7060. The second stage 7060 boosts the voltage of the output signal from the first stage 7040. More specifically, when the second heater enable signal COIL_Z is at a constant logic high level, a third enable signal COIL_X may be activated to boost the output of the first stage 7040. The third enable signal COIL_X is a PWM signal from the controller 2105. The controller 2105 controls the widths of the pulses of the third enable signal COIL_X to boost the output of the first stage 7040 and generate the input voltage signal COIL_OUT. When the third enable signal COIL_X is at a constant low logic level, the output of the second stage 7060 is the output of the first stage 7040.

The second stage 7060 receives the input voltage signal VGATE, the third enable signal COIL_X and the filtered signal GATEON.

The second stage 7060 includes, among other things, an integrated gate driver U7 configured to convert low-current signal(s) from the controller 2105 to high-current signals for controlling switching of transistors of the second stage 7060. The integrated gate driver U7 is also configured to translate voltage levels from the controller 2105 to voltage levels required by the transistors of the second stage 7060. In the example embodiment shown in FIG. 12B, the integrated gate driver U7 is a half-bridge driver. However, example embodiments should not be limited to this example.

In more detail, the input voltage signal VGATE from the boost converter circuit 7020 is input to the second stage 7060 through a filter circuit including a resistor R18 and a capacitor C28. The resistor R18 may have a resistance of 10 ohms and the capacitor C28 may have a capacitance of 1 microfarad.

The filter circuit including the resistor R18 and the capacitor C28 is connected to the VCC pin (pin 4) of the integrated gate driver U7 and the anode of Zener diode DI at node Node14. The second terminal of the capacitor C28 is connected to ground. The anode of the Zener diode D2 is connected to a first terminal of capacitor C27 and a boost pin BST (pin 1) of the integrated gate driver U7 at node Node15. A second terminal of the capacitor C27 is connected to the switching node pin SWN (pin 7) of the integrated gate driver U7 and between transistors Q1 and Q4 at node Node 16.

In the example embodiment shown in FIG. 12C, the Zener diode DI and the capacitor C27 form part of a boot-strap charge-pump circuit connected between the input voltage pin VCC and the boost pin BST of the integrated gate driver U7. Because the capacitor C27 is connected to the input voltage signal VGATE from the boost converter circuit 7020, the capacitor C27 charges to a voltage almost equal to the input voltage signal VGATE through the diode D1. The capacitor C31 may have a capacitance of 220 nanofarads.

Still referring to FIG. 12C, a resistor R21 is connected between the high side gate driver pin DRVH (pin 8) and the switching node pin SWN (pin 7). A gate of the transistor Q4 is connected to the low side gate driver pin DRVL (pin 5) of the integrated date driver U7.

A first terminal of the inductor LA is connected to the output of the first stage 7040 and a second terminal of the inductor LA is connected to the node Node16. The inductor LA serves as the main storage element of the output of the first stage 7040. In example operation, when the integrated gate driver U7 outputs a low level signal from low side gate driver pin DRVL (pin 5), the transistor Q4 switches to a low impedance state (ON), thereby allowing current to flow through inductor LA and transistor Q4. This stores energy in inductor LA, with the current increasing linearly over time. The current in the inductor is proportional to the switching frequency of the transistors (which is controlled by the third heater enable signal COIL_X).

A resistor R10 and a capacitor C29 form a filter circuit connected to the input pin IN (pin 2) of the integrated gate driver U7. The filter circuit is configured to remove high frequency noise from the third heater enable signal COIL_X input to the input pin IN.

A resistor R20 is connected to the filter circuit and the input pin IN at node Node17. The resistor R20 is used as a pull-down resistor, such that if the third heater enable signal COIL_X is floating (or indeterminate), then the input pin IN of the integrated gate driver U7 is held at a logic low level to prevent activation of the heater 336.

A resistor R30 and a capacitor C37 form a filter circuit connected to a pin OD (pin 3) of the integrated gate driver U6. The filter circuit is configured to remove high frequency noise from the input voltage signal VGATE input to the pin OD.

The pin OD of the integrated gate driver U7 receives the filtered signal GATEON.

The transistors Q1 and Q4 field-effect transistors (FETs). A gate of the transistor Q1 and a first terminal of the resistor R21 are connected to the high side gate driver pin DRVH (pin 8) of the integrated gate driver U7 at a node Node18.

A source of the transistor Q1 is connected to a second terminal of the resistor R21, an anode of a Zener diode D3, a drain of the transistor Q4, a first terminal of a capacitor C35, a second terminal of the capacitor C27 and the switching node pin SWN (pin 7) of the integrated gate driver U7 at node Node 16.

A gate of the transistor Q4 is connected to the low side gate driver pin DRVL (pin 5) of the integrated gate driver U7 and a first terminal of a resistor R27 at a node Node19. A source of the transistor Q4 and a second terminal of the resistor R27 are connected to ground.

A second terminal of the capacitor C35 is connected to a first terminal of a resistor R29. A second terminal of the resistor R29 is connected to ground.

A drain of the transistor Q1 is connected to a first terminal of a capacitor C36, a cathode of the Zener diode D3 and a cathode of a Zener diode D5 at a node Node20. A second terminal of the capacitor C36 and an anode of the Zener diode D5 are connected to ground. An output terminal 7065 of the second stage 7060 is connected to the node Node20 and outputs the input voltage signal COIL_OUT. The output terminal 7065 serves as the output of the heating engine control circuit 2127.

The capacitor C35 may be a smoothing capacitor and the resistor limits in-rush current. The Zener diode D3 is a blocking diode to stop a voltage in the node Node20 discharging into the capacitor C35. The capacitor C36 is an output capacitor charged by the second stage 7060 (and reduces ripple in COIL_OUT) and the Zener diode D5 is an ESD (electrostatic discharge) protection diode.

When the low side gate drive signal output from the low side gate driver pin DRVL is high, the transistor Q4 is in a low impedance state (ON), thereby connecting the node Node16 to ground and increasing the energy stored in the magnetic field of the inductor LA.

As mentioned above, because the capacitor C27 is connected to the input voltage signal VGATE from the boost converter circuit 7020, the capacitor C27 charges to a voltage equal or substantially equal to the input voltage signal VGATE through the diode D1.

When the low side gate drive signal output from the low side gate driver pin DRVL is low, the transistor Q4 switches to the high impedance state (OFF), and the high side gate driver pin DRVH (pin 8) is connected internally to the bootstrap pin BST within the integrated gate driver U7. As a result, transistor Q1 is in a low impedance state (ON), thereby connecting the switching node SWN to the inductor LA.

In this case, the node Node15 is raised to a bootstrap voltage V(BST)≈V(VGATE)+V(INDUCTOR), which allows the gate-source voltage of the transistor Q1 to be the same or substantially the same as the voltage of the input voltage signal VGATE (e.g., V(VGATE)) regardless (or independent) of the voltage from the inductor L4. As the second stage 7060 is a boost circuit, the bootstrap voltage may also be referred to as a boost voltage.

The switching node SWN (Node 8) is connected to the inductor voltage and the output capacitor C36 is charged, generating the voltage output signal COIL_OUT (the voltage output to the heater 336) that is substantially independent of the voltage output from the first stage 7040.

FIGS. 13A-13B illustrate methods of controlling a heater in a non-combustible aerosol-generating device according to example embodiments.

Many non-combustible devices use a preheat of organic material (e.g., tobacco) prior to use. The preheat is used to elevate the temperature of the material to a point at which the compounds of interest begin to volatize such that the first negative pressure applied by an adult operator contains a suitable volume and composition of aerosol.

In at least some example embodiments, applied energy is used as a basis for controlling the heater during preheat. Using applied energy to control the heater improves the quality and consistency of the first negative pressure applied by the adult operator. By contrast, time and temperature are generally used as a basis for controlling the preheat.

The methods of FIGS. 13A-13B may be implemented at the controller 2105. In one example, the methods of FIGS. 13A-13B may be implemented as part of a device manager Finite State Machine (FSM) software implementation executed at the controller 2105.

As shown in FIG. 13A, the method includes increasing a temperature of a heater to a preheat temperature at S1300. The controller may increase the temperature of the heater to the preheat temperature by initially applying a maximum power. An example embodiment of S1300 is further illustrated in FIG. 13B.

As shown in FIG. 13B, the controller detects that a capsule is inserted into the aerosol-generating device at S1320. In some example embodiments, the controller obtains a signal from an opening closing switch coupled to the door, which is illustrated in FIGS. 1-2 and 4-5. In other example embodiments, the aerosol-generating device further includes (or alternatively includes) a capsule detection switch. The capsule detection switch detects whether the capsule is properly inserted (e.g., capsule detection switch gets pushed down/closes when the capsule is properly inserted). Upon the capsule being properly inserted, the controller may generate the signal PWR_EN_VGATE (shown in FIG. 12A) as a logic high level. In addition, the controller may perform a heater continuity check to determine the capsule is inserted and the heater resistance is within the specified range (e.g. ±20%).

After a capsule has been inserted (as detected by the switch) and/or when the aerosol-generating device 100 is turned on (e.g. by operation of the button), the heater 336 may be powered with a low power signal from the heating engine control circuit (˜1 W) for a short duration (˜50 ms) and the resistance may be calculated from the measured voltage and current during this impulse of energy. If the measured resistance falls within the range specified (e.g. a nominal 2100 m Ω±20%) the capsule is considered acceptable and the system may proceed to aerosol-generation.

The low power and short duration is intended to provide a minimum amount of heating to the capsule (to prevent any generation of aerosol).

Upon detecting that the capsule is inserted at S1320, the controller obtains operating parameters from the memory. The operating parameters may include values identifying a maximum power level (Pₘₐₓ), a preheat temperature and a preheat energy threshold. For example, the operating parameters may be predetermined based on empirical data or adjusted based on obtained measurements from the capsule (e.g., voltage and current). However, example embodiments are not limited thereto. In addition to or alternatively, the operating parameters may include different preheat temperatures for subsequent instances for a multi-instances device. For example, the controller may obtain operating parameters for an initial instance and operating parameters for a second subsequent instance. In addition, the controller sets a puff count (e.g., number of instances a negative pressure exceeds a threshold/number of detected puffs) to zero.

At S1325, the controller determines whether a preheat has started. In some example embodiments, the controller may start the preheat upon receiving an input from the on-product controls indicating a consumer has pressed a button to initiate the preheat. In some example embodiments, the button may be separate from a button that powers on the aerosol-generating device and in other example embodiments, the button may be the same button that powers on the aerosol-generating device. In other example embodiments, the preheat may be started based on another input such as sensing an airflow above a threshold level, motion of the device (e.g. sensed by an accelerometer), or upon sensing that a capsule has been inserted. In other example embodiments, the on-product controls may permit an adult operator to select one or more temperature profiles (each temperature profile associated operating parameters stored in the memory).

If the controller determines that no preheat has started, the method proceeds to S1330 where the controller determines whether an off timer has elapsed. If the off timer has not elapsed, the method returns to S1330 and if the controller determines the off timer has elapsed, the controller causes the aerosol-generating device to display an “off” state and power off at S1335. The off timer starts when the detected air flow falls below a threshold level. The off timer is used to display the “off” state based on inaction for a period of time such as 15 minutes. However, example embodiments are not limited to 15 minutes. For example, the duration of the off timer may be 2 minutes or 10 minutes.

If the controller determines the preheat has started (e.g., detects input from the on-product controls) at S1325, the controller obtains the operating parameters associated with the input from the on-product controls from the memory at S1340. In an example, where the aerosol-generating instance is not the initial instance for the capsule, the controller may obtain operating parameters associated with the instance number. For example, the memory may store different temperature targets based on the instance number and/or puff count (e.g., different temperature targets for instance numbers, respectively) and different target energy levels to use for preheating based on the instance number.

The initial instance occurs when the controller initiates the preheat algorithm for a first time after detecting a capsule has been removed and one has since been inserted. Additionally, the instance number increments if the instance times out (e.g. after 8 minutes) or if the consumer switches off the device during an instance.

Upon obtaining the operating parameters at S1340, the controller may cause the aerosol-generating device to display an indication that preheat has started via the aerosol indicators.

At S1345, the controller ramps up to a maximum available power to the heater (through the VGATE, COIL_Z and COIL_X signals provided to the heating engine control circuit 2127) (e.g., the controller provides a maximum available power of 10 W within 200 ms). In more detail, the controller requests maximum power, but ramps up to the maximum power to reduce an instantaneous load on the power supply. In an example embodiment, the maximum available power is a set value based on the capability of a battery and to minimize overshoot such that the aerosol-forming substrate is not burnt by the heater (i.e., how much energy can be put into the aerosol-forming substrate without burning). The maximum available power may be set based on empirical evidence and may be between 10-15 W. The controller provides the maximum available power until the controller determines that a preheat temperature of the heater (e.g., 280° C.) is approaching, at S1345. While 280° C. is used as an example preheat temperature for a heater, it should be understood example embodiments are not limited thereto. For example, the preheat temperature of the heater may be less than 400° C., such as 350° C. Moreover, the preheat temperature may be based on the materials in the aerosol-forming substrate.

The controller may determine the temperature of the heater using the measured voltages from the heater voltage measurement circuit (e.g., COIL_VOL) and the compensation voltage measurement circuit, and may determine the measured current from the heater current measurement circuit (e.g., COIL_RTN_I). The controller may determine the temperature of the heater 336 in any known manner (e.g., based on the relatively linear relationship between resistance and temperature of the heater 336).

Further, the controller may use the measured current COIL_RTN_I and the measured voltage COIL_RTN to determine the resistance of the heater 336, heater resistance R_{Heater} (e.g., using Ohm's law or other known methods). For example, according to at least some example embodiments, the controller may divide the measured voltage COIL_RTN (or compensated voltage VCOMP) by the measured current COIL_RTN_I to be the heater resistance R_{Heater}.

In some example embodiments, the measured voltage COIL_RTN measured at the measurement contacts for the resistance calculation may be used in temperature control.

For example, the controller 2105 may use the following equation to determine (i.e., estimate) the temperature:

$${R}_{Heater} = {R}_{0} [ 1 + ▯ ( T - {T}_{0} ) ]$$

where □ is the temperature coefficient of resistance (TCR) value of the material of the heater, R₀ is a starting resistance and T₀ is a starting temperature, R_{Heater} is the current resistance determination and T is the estimated temperature.

The starting resistance R₀ is stored in the memory 2130 by the controller 2105 during the preheat. More specifically, the controller 2105 may measure the starting resistance R₀ when the power applied to the heater 336 has reached a value where a measurement error has a reduced effect on the temperature calculation. For example, the controller 2105 may measure the starting resistance R₀ when the power supplied to the heater 336 is 1 W (where resistance measurement error is approximately less than 1%). However, such a wattage to measure the starting resistance Ro is not limited to 1 W.

The starting temperature T₀ is the ambient temperature at the time when the controller 2105 measures the starting resistance R₀. The controller 2105 may determine the starting temperature T₀ using an onboard thermistor to measure the starting temperature T₀ or any temperature measurement device. In other example embodiments, the starting temperature T₀ may be a fixed value such as 25° C.

According to at least one example embodiment, a 10 ms (millisecond) measurement interval may be used for measurements taken from the heater current measurement circuit 21258 and the heater voltage measurement circuit 21252 (since this may be the maximum sample rate). In at least one other example embodiment, however, for a resistance-based heater measurement, a 1 ms measurement interval (the tick rate of the system) may be used.

In other example embodiments, the determining of the heater temperature value may include obtaining, from a look-up table (LUT), based on the determined resistance, a heater temperature value. In some example embodiments, a LUT indexed by the change in resistance relative to a starting resistance may be used.

The LUT may store a plurality of temperature values that correspond, respectively, to a plurality of heater resistances, the obtained heater temperature value may be the temperature value, from among the plurality of temperature values stored in the LUT, that corresponds to the determined resistance.

Additionally, the aerosol-generating device 100 may store (e.g., in the memory 2130) a look-up table (LUT) that stores a plurality of heater resistance values as indexes for a plurality of respectively corresponding heater temperature values also stored in the LUT. Consequently, the controller may estimate a current temperature of the heater 336 by using the previously determined heater resistance RHeater as an index for the LUT to identify (e.g., look-up) a corresponding heater temperature T from among the heater temperatures stored in the LUT.

Once the controller determines the target preheat temperature is approaching, the controller begins to reduce the applied power to the heater to avoid a temperature overshoot.

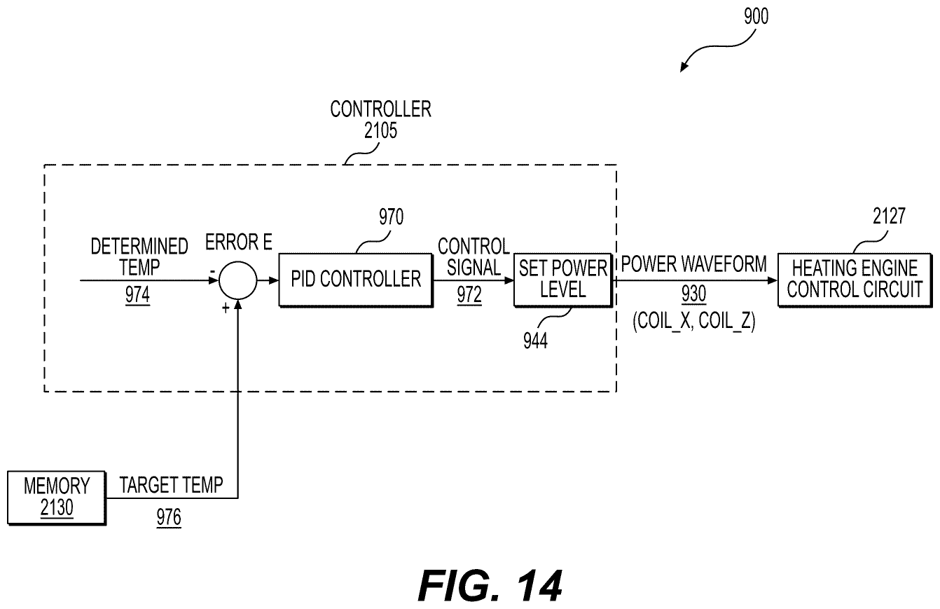

A proportional-integral-derivative (PID) controller (shown in FIG. 14) applies a proportionate control based on an error signal (i.e., the target temperature minus the current determined temperature) so, as the error signal reduces towards zero, the controller 2105 starts to back off the power being applied (this is largely controlled by a proportional term (P) of the PID controller, but an integral term (I), and a derivative term also contribute).

The P, I and D values balance overshoot, latency and steady state error against one another and control how the PID controller adjusts its output. The P, I and D values may be derived empirically or by simulation.

FIG. 14 illustrates a block diagram illustrating a temperature heating engine control algorithm according to at least some example embodiments.

Referring to FIG. 14, the temperature heating engine control algorithm 900 uses a PID controller 970 to control an amount of power applied to the heating engine control circuit 2127 so as to achieve a desired temperature. For example, as is discussed in greater detail below, according to at least some example embodiments, the temperature heating engine control algorithm 900 includes obtaining a determined temperature value 974 (e.g., determined as described above); obtaining a target temperature value 976 (e.g., the preheat temperature, the first heating temperature, the second heating temperature, etc.) from the memory 2130; and controlling, by a PID controller (e.g., PID controller 970), a level of power provided to the heater, based on the determined heater temperature value and the target temperature value.

Further, according to at least some example embodiments, the target temperature 976 serves as a setpoint (i.e., a temperature setpoint) in a PID control loop controlled by the PID controller 970. In some example embodiments, each puff is associated with a temperature setpoint. After a puff is detected, the controller may heat the heater to a target temperature associated with a subsequent puff prior to the subsequent puff occurring.

Consequently, the PID controller 970 continuously corrects a level of a power control signal 972 so as to control a power waveform 930 (i.e., COIL_X and COIL_Z) output by the power level setting operation 944 to the heating engine control circuit 2127 in such a manner that a difference (e.g., a magnitude of the difference) between the target temperature 976 and the determined temperature 974 is reduced or, alternatively, minimized. The difference between the target temperature 976 and the determined temperature 974 may also be viewed as an error value which the PID controller 970 works to reduce or minimize.

For example, according to at least some example embodiments, the power level setting operation 944 outputs the power waveform 930 such that levels of the power waveform 930 are controlled by the power control signal 972. The heating engine control circuit 2127 causes an amount of power provided to the heater 336 by the power supply 1234 to increase or decrease in manner that is proportional to an increase or decrease in a magnitude of the power levels of a power level waveform output to the heating engine control circuit 2127. Consequently, by controlling the power control signal 972, the PID controller 970 controls a level of power provided to the heater 336 (e.g., by the power supply 1234) such that a magnitude of the difference between a target temperature value (e.g., target temperature 976) and a determined temperature value (e.g., determined temperature 974) is reduced, or alternatively, minimized.

According to at least some example embodiments, the PID controller 970 may operate in accordance with known PID control methods. According to at least some example embodiments, the PID controller 970 may generate 2 or more terms from among the proportional term (P), the integral term (I), and the derivative term (D), and the PID controller 970 may use the two or more terms to adjust or correct the power control signal 972 in accordance with known methods. In some example embodiments, the same PID settings for the initial and subsequent heating phases may be used. A preheat phase may be considered the period when heating occurs before a first puff is detected and a heating phase may be considered the time between a puff ending and a subsequent puff beginning.

In some example embodiments, PID terms may be different between the preheating and temperature phases and puff phases. For example, the P term may be 100, the I term may be zero and the D term may be zero for the preheat phase and subsequent heating phases. During a puff phase, the P term may be 500, the I term may be one and the D term may be zero.

In other example embodiments, different PID settings may be used for each phase (e.g., if the temperature targets used for the initial and subsequent heating phases are substantially different). For example, the PID settings may be changed as the aerosol-forming substrate in the capsule becomes more depleted (e.g., the P term may be reduced).

FIGS. 15B and 16B illustrate examples in which levels of the power waveform 930 may vary over time as the PID controller 970 continuously corrects the power control signal 972 provided to the power level setting operation 944. FIGS. 15B and 16B shows an example manner in which levels of the power waveform 930 may vary as target temperatures and energy thresholds are reached. The power in FIGS. 15B and 16B is COIL_VOL*COIL_CUR.

In FIGS. 15B and 16B, the PID loop will start to lower the applied power from a jump in power as the temperature approaches the setpoint, which reduces overshoot of the target temperature.

FIGS. 15B and 16B is discussed in further detail below.

Referring back to FIG. 13B, the controller determines an estimated energy that has been delivered to the heater as part of heating the heater to the preheat temperature, at S1350.

As shown in FIG. 13B and previously discussed, the controller controls power supplied to the heater at S1345. At S1350, the controller determines whether an estimated energy applied to the heater has reached a preheat energy threshold. More specifically, the controller integrates (or sums the samples) the power delivered to the heater since starting the preheat to estimate the energy delivered to the heater. In an example embodiment, the controller determines the power (Power=COIL_VOL*COIL_CUR) applied to the heater every millisecond and uses that determined power as part of the integration (or the sum).

If the controller determines the preheat energy threshold has not been met, the method continues to monitor the preheat energy at S1350.

In some example embodiments, when the controller determines the applied energy reaches the preheat energy threshold (e.g., 75J), the controller causes the aerosol-generating device may output a preheat complete indication at via the aerosol indicators to indicate to the adult consumer that the preheat is complete.

Referring to both FIGS. 13A and 13B, the controller decreases the temperature of the heater to a first target heating temperature at S1305. The first target heating temperature (i.e., first temperature setpoint) is associated with a puff count of zero.

To heat the heater to the first target heating temperature, the controller reduces power to the heater using the temperature control algorithm described in FIG. 14. The first target heating temperature is a different temperature setpoint than the target preheat temperature.

In some example embodiments, when the controller determines that the heater has reached or decreased below the first target heating temperature plus an offset (e.g., the first temperature setpoint plus five percent), the controller causes the aerosol-generating device to output a ready to use indication via the aerosol indicators to indicate to the adult consumer that the device is ready to use. In some example embodiments, the preheat completion indication is output and, subsequently, the ready to use indication is output. In other example embodiments, the aerosol-generating device outputs the ready to use indication and does not output the preheat completion indication.

An adult operator may start aerosol-generation after the preheat temperature target is reached. More specifically, the controller 2105 may initiate aerosol-generation (i.e., supplying power to the heater such that the heater reaches a temperature sufficient to produce an aerosol) upon detecting a negative pressure being applied by the adult operator and upon the preheat temperature target being reached.

During operation, an airflow is detected by the airflow sensor.

The controller monitors input from the airflow sensor to determine whether the detected airflow exceeds a first airflow threshold at S1355. An airflow exceeding a first airflow threshold indicates a puff occurring. If the controller determines the detected airflow does not exceed the first airflow threshold, the controller determines whether a threshold time has elapsed such that the device would enter a shutdown (or also referred to as sleep) mode or go into an idle mode at S1357. If the threshold time has not lapsed, the method returns to S1355 and the controller continues to control the temperature of the heater in accordance with the temperature target.

While a time is used in the example of FIG. 13B to determine whether to turn off the aerosol-generating device and/or enter an idle mode, it should be understood that other criteria may be also be used. For example, the controller may determine to prohibit aerosol-generating after a threshold time of exceeding a threshold negative pressure or instance energy consumption has occurred. Further, it should be understood that pressure values and thresholds may be used instead of airflow values and thresholds.

The airflow sensor provides a measurement in magnitude every 20 ms to the controller 2105. The controller uses a threshold detection that is averaged over a number of samples (e.g., 7 samples) to reduce noise. For example, the controller averages a number of sample magnitudes from the airflow sensor and determines whether the average exceeds the first airflow threshold (e.g., 1 ml/s).

In some example embodiments, the controller determines that a sufficient negative pressure is being applied to the aerosol-generating device to initiate an aerosol-generating event (i.e., a puff) when the detected airflow is equal to the first threshold and in other example embodiments, the controller determines that a sufficient negative pressure is not being applied to the aerosol-generating device to initiate an aerosol-generating event when the detected airflow is equal to the first threshold.

If the controller determines the detected airflow exceeds the first threshold at S1355, the controller increases the puff count and lowers the power supply to the heater. In some example embodiments, the controller stops supplying power to the heater when the end of the puff is detected. For example, the controller may set the set the duty cycle of the PWM to zero for a period of time (e.g., predetermined) when the end of the puff is detected, even though a target power may be above zero. After the period time, the controller then controls the PWM in accordance with the target power associated with the temperature setpoint. In an example, the period of time may be 1 ms, but is no limited thereto.

The method proceeds to S1360. At S1360, the controller determines whether the puff count is equal to a maximum puff count (i.e., a last puff). If the controller determines the puff count is equal to a maximum puff count, the controller shuts power off to the heater at S1370 and the device turns off at S1335.

FIG. 13C illustrates an alternative embodiment using separate timers for an idle mode and a power off mode. The timer used at S1357 may be referred to as a first timer and a timer used for the idle mode may be referred to as a second timer. Both the first timer and the second timer may be started when capsule insertion is detected at S1320.

If a puff is not detected at S1355, the controller then determines whether the second timer has expired. The second timer may be a fixed time (e.g., 60 s). In some example embodiments, the period of time of the time of the second timer may be a time calculated by the controller based on a previous usage pattern (e.g. at least one of longest measured time to first Puff, longest inter-Puff interval, or longest time Device has been stationary). If the second timer has not expired, the controller continues to monitor whether a puff has been detected at S1355.

If the controller determines that the second timer has expired, the controller causes the device to enter an idle mode by decreasing the temperature of the heater to an idle temperature setpoint at S1392. The idle temperature setpoint is a temperature setting to conserve volatile content in the capsule and to reduce battery consumption. The idle temperature setpoint may be a fixed value (e.g. 120° C.). In some example embodiments, the idle temperature setpoint may be a ratio of the current temperature setpoint associated with the puff count (e.g., 50% of the temperature setpoint associated with the puff count) or a fixed offset to the current temperature setpoint associated with the puff count (e.g. 100° C. less than the temperature setpoint associated with the puff count). When the idle temperature setpoint is based on the current temperature setpoint associated with the puff count, the device takes into account a current usage state of the Capsule (i.e., a higher idle temperature setting is used if the capsule is partially depleted).

At S1357, the controller determines whether the first time is expired. If the first timer is expired, the controller prohibits power to the heater at S1370. If the controller determines that the first time has not expired, the controller determines whether an activity associated with the device has been detected at S1394.

Activity may be detected by a movement sensor such as an accelerometer or a change in pressure or rate of change of pressure measured by an ambient pressure sensor. In some example embodiments, the activity may be a puff start, a device movement or a button actuation. The movement sensor might be an accelerometer or a change in pressure or rate of change of pressure measured by an ambient pressure sensor. The detected activity might be a Puff start, a Device movement, a button actuation or connection to a charger.

If no activity is detected, the controller returns to S1357. If activity is detected, the controller increases the temperature of the heater to the temperature setpoint when the device entered the idle mode (i.e., the temperature setpoint associated with the puff count) and resets the second timer at S1396. The controller then monitors whether a puff has occurred at S1355.

It should be understood that, when the controller detects a puff or an activity (as described above with reference to S1394), the controller resets the second timer. Consequently, if the controller detects a puff at S1355, the controller resets the second timer.

Referring back to FIG. 13B, if the puff count is less than the maximum puff count at S1360, the controller waits until the puff is over at S1373 (e.g., the measured airflow falls below an airflow threshold indicating a puff is no longer occurring) and then determines the temperature setpoint associated with the puff count at S1375. It should be understood that the airflow threshold indicating a start of a puff and the airflow threshold indicating an end of a puff may be the same or different as described in U.S. application Ser. No. 17/151,409, filed Jan. 18, 2021, the entire contents of which are herein incorporated by reference.

If the current temperature setpoint is associated with the puff count, the controller does not change the temperature setpoint and the method proceeds back to S1355. If the temperature setpoint associated with the puff count is not the current temperature setpoint, the controller changes the current temperature setpoint to be the temperature setpoint associated with the puff count and heats the heater in accordance with the temperature setpoint associated with the subsequent puff at S1380. The puff count may be managed as disclosed in U.S. application Ser. No. 17/947,334, filed Sep. 19, 2022, the entire contents of which are herein incorporated by reference. The method then proceeds back to S1355.

A LUT may store the correspondence between the temperature setpoints and puff counts. According to at least one example embodiment, each temperature setpoint associated with a puff is greater than or equal to a temperature setpoint associated with a previous puff. According to at least one example embodiment, at least two sequential puffs are associated with the same temperature setpoint.

FIGS. 15A-15B illustrate a temperature profile and target power profile of a heater (e.g., the heater 336) using the methods shown in FIGS. 13A-13B. In the examples shown in FIGS. 15A-15B, a last target temperature Tn is high than a preheat temperature Tpre.

FIG. 15A illustrates a temperature profile of the heater according to the puff count and FIG. 15B illustrates a target power profile overlaid on the temperature profile shown in FIG. 15A. The example shown in FIGS. 15A-15B has a preheating temperature setpoint and 7 heating temperature setpoints for a maximum puff count of 14. However, it should be understood that a different number of preheating temperature setpoints and heating temperature setpoints may be used. Moreover, a maximum puff count may be greater than or less than 14.

Referring to FIGS. 15A-15B, at to, the controller receives an input regarding initiation of a preheat which causes the controller to ramp up power to apply a maximum power Pmax to the heater to reach the target preheat temperature Tpre. As described above, when approaching a target temperature, the controller reduces the power applied to the heater to reduce the likelihood of temperature overshoot and then maintains the target preheat temperature Tpre. At ten, the controller determines that the preheat energy has been reached and sets the current temperature setpoint to the first target heating temperature T1. The first target heating temperature T1 corresponds to a puff count of zero.

At t1, the controller detects an airflow (or pressure or pressure change) sufficient to initiate an aerosol-generating event (i.e., a puff is detected) and lowers the power supplied to the heater, which may be the minimum power Pmin in some example embodiments.

At t2, the controller determines detects that the airflow is no longer sufficient for an aerosol-generating event (i.e., the puff has ended). The controller then determines the temperature setpoint associated with the puff count being one. In the example shown in FIGS. 15A-15B, the temperature setpoint associated with the puff count being one is the first target heating temperature T1 (i.e., the same temperature setpoint associated with the puff count being zero). Thus, the controller supplies power to the heater to increase the temperature back to the first target heating temperature T1 due to the drop in temperature from the puff. The controller then supplies power to the heater to maintain the temperature at the first target heating temperature T1 until a second puff is detected.

At t3, the controller detects an airflow (or pressure or pressure change) sufficient to initiate an aerosol-generating event (i.e., a puff is detected) and lowers the power supplied to the heater to the minimum power Pₘᵢₙ in some example embodiments.

At t4, the controller determines detects that the airflow is no longer sufficient for an aerosol-generating event (i.e., the puff has ended).

The controller then determines the temperature setpoint associated with the puff count being two. In the example shown in FIGS. 15A-15B, the temperature setpoint associated with the puff count being two is a second target heating temperature T2 (i.e., a different temperature setpoint associated with the puff count being zero). The controller sets the current temperature setpoint to the second target heating temperature T2 and supplies power to the heater to increase the temperature the second target heating temperature T2. The controller then supplies power to the heater to maintain the temperature at the second target temperature T2 until a third puff is detected.

The process continues until the maximum puff count is reached. As shown, the temperature setpoints increase as the puff count increases. More specifically, each of the temperature setpoints (i.e., target heating temperatures T1-Tn) is higher than an immediate preceding temperature setpoint and lower than an immediately subsequent temperature setpoint. For example, the target heating temperature T4 is greater than the target heating temperature T3 and less than the target heating temperature T5.

In the example shown in FIGS. 15A-15B, the first target heating temperature T1 is between 235° C. and 240° C. The final target heating temperature Tn may be higher than the target preheating temperature, for example 290° C. The temperature setpoints may be determined based on sensory and analytical data to obtain consistency for a threshold number of puffs for a capsule (e.g., a maximum number of puffs). For example, the change between adjacent temperature setpoints (e.g., a first heating temperature and a subsequent second heating temperature) may be 5-10° C.

FIG. 16A illustrates a temperature profile of the heater according to the puff count and FIG. 16B illustrates a target power profile overlaid on the temperature profile shown in FIG. 16A. The process of performing the example shown in FIGS. 16A-16B is the same as FIGS. 15A-15B except the heating temperature setpoints are different and the example shown in FIGS. 16A-16B have less heating temperature setpoints.

In the example shown in FIGS. 16A-16B, the first target heating temperature T1 is between 235° C. and 240° C. The final target heating temperature Tn may be the same as or lower than the target preheating temperature, for example 280° C.

While some example embodiments have been disclosed herein, it should be understood that other variations may be possible. Such variations are not to be regarded as a departure from the spirit and scope of the present disclosure, and all such modifications as would be obvious to one skilled in the art are intended to be included within the scope of the following claims.

Although described with reference to specific examples and drawings, modifications, additions and substitutions of example embodiments may be variously made according to the description by those of ordinary skill in the art. For example, the described techniques may be performed in an order different with that of the methods described, and/or elements such as the described system, architecture, devices, circuit, and the like, may be connected or combined to be different from the above-described methods, or results may be appropriately achieved by other elements or equivalents.

We claim:

1. An aerosol-generating device comprising:
a sensor configured to detect at least one puff; and
processing circuitry configured to cause the aerosol-generating device to,
increase a temperature of a heater to a preheat temperature,
decrease the temperature of the heater to a first heating temperature before a first detected puff of the detected puffs, the first heating temperature being below the preheat temperature, and
increase the temperature of the heater based on a number of the at least one detected puff.

2. The aerosol-generating device of claim 1, wherein each puff of the at least one detected puff is associated with a temperature setpoint.

3. The aerosol-generating device of claim 1, wherein the processing circuitry is configured to cause the aerosol-generating device to increase the temperature of the heater to a second heating temperature after a first number of the at least one detected puff, the first number being greater than one.

4. The aerosol-generating device of claim 3, wherein the second heating temperature is greater than the first heating temperature and less than the preheat temperature.

5. The aerosol-generating device of claim 4, wherein the at least one detected puff is a plurality of detected puffs and the processing circuitry is configured to cause the aerosol-generating device to maintain the first heating temperature until a second number of the plurality of detected puffs and increase the temperature of the heater to a third heating temperature after the second number of the plurality of detected puffs, the second number of the plurality of detected puffs being greater than two.

6. The aerosol-generating device of claim 1, wherein the processing circuitry is configured to cause the aerosol-generating device to increase the temperature of the heater when the number of the at least one detected puff reaches a predetermined number.

7. The aerosol-generating device of claim 6, wherein the processing circuitry is configured to cause the aerosol-generating device to increase the temperature of the heater to a final heating temperature when the number of the at least one detected puff reaches the predetermined number.

8. The aerosol-generating device of claim 7, wherein the processing circuitry is configured to cause the aerosol-generating device to maintain the temperature of the heater at the final heating temperature until the number of the at least one detected puff reaches a maximum number.

9. The aerosol-generating device of claim 8, wherein the final heating temperature is less than or equal to the preheat temperature.

10. The aerosol-generating device of claim 8, wherein the final heating temperature is greater than the preheat temperature.

11. The aerosol-generating device of claim 1, wherein the processing circuitry is configured to cause the aerosol-generating device to,
reduce a power to the heater to a first power during at least one of the detected puffs.

12. The aerosol-generating device of claim 11, wherein the at least one detected puff is a plurality of detected puffs and the processing circuitry is configured to cause the aerosol-generating device to,
supply a second power to the heater for a period of time between adjacent puffs of the plurality of detected puffs, the second power being greater than the first power.

13. The aerosol-generating device of claim 12, wherein the processing circuitry is configured to cause the aerosol-generating device to,
supply the second power to the heater for the period of time using a proportional-integral-derivative (PID) controller, wherein the processing circuitry is configured to cause the aerosol-generating device to change at least one of a proportional term, an integral term and a derivative term of the PID controller.

14. The aerosol-generating device of claim 13, wherein the processing circuitry is configured to cause the aerosol-generating device to maintain values of the proportional term, the integral term and the derivative term of the PID controller during the period of time.

15. The aerosol-generating device of claim 1, wherein the processing circuitry is configured to cause the aerosol-generating device to,
determine a voltage applied to the heater and a current applied to the heater over a period of time, and
decrease the temperature of the heater to the first heating temperature based on the voltage applied to the heater and the current applied to the heater over the period of time.

16. The aerosol-generating device of claim 15, wherein the processing circuitry is configured to cause the aerosol-generating device to,
determine a sum of products of the voltage applied to the heater and the current applied to the heater, and
determine if the sum is greater than a threshold, wherein the decrease of the temperature of the heater to the first heating temperature occurs when the sum is greater than the threshold.

17. The aerosol-generating device of claim 1, wherein the aerosol-generating device is configured to receive a capsule containing an aerosol-forming substrate to be heated by the heater.

18. The aerosol-generating device of claim 17, wherein the heater is in the capsule.

19. The aerosol-generating device of claim 1, wherein the heater is external to the capsule.

20. A method of generating aerosol in an aerosol-generating device, the method comprising:
increasing a temperature of a heater of the aerosol-generating device to a preheat temperature;
decreasing the temperature of the heater to a first heating temperature;
detecting at least one puff after decreasing the temperature of the heater to the first heating temperature; and
increasing the temperature of the heater based on a number of the at least one detected puff.
